SEQUENCE LISTING (19 sequences)

SEQ ID NO: 1 - RNA, 22 nt, Homo sapiens
  source 1..22: mol_type = other RNA
tgaggtagtaggttgtgtggtt

SEQ ID NO: 2 - RNA, 23 nt, Homo sapiens
  source 1..23: mol_type = other RNA
caaagtgcttacagtgcaggtag

SEQ ID NO: 3 - RNA, 23 nt, Homo sapiens
  source 1..23: mol_type = other RNA
tgtgcaaatccatgcaaaactga

SEQ ID NO: 4 - RNA, 23 nt, Homo sapiens
  source 1..23: mol_type = other RNA
caaagtgctcatagtgcaggtag

SEQ ID NO: 5 - RNA, 22 nt, Homo sapiens
  source 1..22: mol_type = other RNA
tgtaaacatccccgactggaag

SEQ ID NO: 6 - RNA, 23 nt, Homo sapiens
  source 1..23: mol_type = other RNA
tggagacgcggccctgttggagt

SEQ ID NO: 7 - RNA, 24 nt, Homo sapiens
  source 1..24: mol_type = other RNA
tttggcaatggtagaactcacact

SEQ ID NO: 8 - RNA, 22 nt, Homo sapiens
  source 1..22: mol_type = other RNA
aaaagctgggttgagagggcaa

SEQ ID NO: 9 - RNA, 23 nt, Homo sapiens
  source 1..23: mol_type = other RNA
atcaacagacattaattgggcgc

SEQ ID NO: 10 - RNA, 22 nt, Homo sapiens
  source 1..22: mol_type = other RNA
tttgttcgttcggctcgcgtga

SEQ ID NO: 11 - RNA, 21 nt, Homo sapiens
  source 1..21: mol_type = other RNA
taaagtgctgacagtgcagat

SEQ ID NO: 12 - RNA, 22 nt, Homo sapiens
  source 1..22: mol_type = other RNA
tgtgactggttgaccagagggg

SEQ ID NO: 13 - RNA, 22 nt, Homo sapiens
  source 1..22: mol_type = other RNA
ttgtacatggtaggctttcatt

SEQ ID NO: 14 - RNA, 22 nt, Homo sapiens
  source 1..22: mol_type = other RNA
atgcacctgggcaaggattctg

SEQ ID NO: 15 - RNA, 22 nt, Homo sapiens
  source 1..22: mol_type = other RNA
ctgcagactcgacctcccaggc

SEQ ID NO: 16 - RNA, 21 nt, Homo sapiens
  source 1..21: mol_type = other RNA
acaggagtgggggtgggacat

SEQ ID NO: 17 - RNA, 21 nt, Homo sapiens
  source 1..21: mol_type = other RNA
atgtcccacccccactcctgt

SEQ ID NO: 18 - RNA, 22 nt, Homo sapiens
  source 1..22: mol_type = other RNA
gtcatacacggctctcctctct

SEQ ID NO: 19 - RNA, 22 nt, Homo sapiens
  source 1..22: mol_type = other RNA
tctcactgtagcctcgaacccc

(19) United States
(12) Patent Application Publication
(10) Pub. No.: US 2026/0297682 A1
(43) Pub. Date: Oct. 1, 2026

(54) USE OF CIRCULATING MICRORNA PROFILES FOR IDENTIFICATION OF BRCA1 OR BRCA2 MUTATIONS

(71) Applicants: Dana-Farber Cancer Institute, Inc., Boston, MA (US); The Brigham and Women's Hospital, Inc., Boston, MA (US); Medical University of Lodz, Lodz (PL)

(72) Inventors: Dipanjan Chowdhury, Boston, MA (US); Kevin M. Elias, Boston, MA (US); Wojciech Fendler, Lodz (PL); James Webber, Boston, MA (US); Urszula Smyczynska, Lodz (PL)

(21) Appl. No.: 19/480,782

(22) PCT Filed: May 2, 2024
(86) PCT No.: PCT/US2024/027522

§ 371 Date: Oct. 31, 2025

(30) Foreign Application Priority Data
May 2, 2023 (EP) 23461572.2

(51) Int. Cl. C12Q 1/6886; G16B 20/20; G16B 40/30

(52) U.S. Cl. CPC C12Q 1/6886; G16B 20/20; G16B 40/30; C12Q 2600/118; C12Q 2600/156; C12Q 2600/178

(57) ABSTRACT

A method of predicting a lifetime risk of having one or more cancer, in a subject suspected of having a BRCA 1 or BRCA2 mutation, including: obtaining a sample collected from the subject; determining the amounts of a circulating microRNA selected from the group consisting of: hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19); comparing the amounts of circulating microRNA as determined in step (b) to a statistical model; and identifying the presence of at least one mutation in the BRCA1 or BRCA2 gene.

### STATEMENT REGARDING FEDERALLY SPONSORED RESEARCH OR DEVELOPMENT

This invention was made with government support under grant numbers 1P50CA240243-01A1, 1R03CA283252-01, and 5P50CA240243-04 awarded by the National Institute of Health. The government has certain rights in the invention.

### CROSS-REFERENCE TO RELATED APPLICATIONS

This application claims priority to European application number EP 23461572.2, filed May 2, 2023, the contents of which are incorporated by reference in its entirety.

### SEQUENCE LISTING

The present application contains, as a separate part of the disclosure, a Sequence Listing in XML format. The file name is: 129319.01021_SL_ST26.xml, created on Apr. 30, 2024, 17,745 bytes, which is incorporated by reference herein, in its entirety.

### FIELD

The present disclosure provides a method of using novel circulating miRNA profiles for the identification of BRCA1 or BRCA2 mutations, and predicting lifetime risk of developing cancer.

### BACKGROUND

Hereditary breast and ovarian cancer (HBOC) is the most common hereditary cancer syndrome, and the two most commonly mutated genes in HBOC, BRCA1 and BRCA2, both play critical roles in mediating DNA repair through homologous recombination (HR). See Shulman, (2010). Germline mutations in BRCA1 or BRCA1 (BRCA1/2) account for 10%-15% of ovarian cancers, 5%-10% of breast cancers, and 3%-5% of pancreatic and prostate cancers. Loss of HR, known as HR deficiency (HRD), impairs the ability of cells to repair double-strand DNA breaks, leaving cells vulnerable to mutagenesis from ionizing radiation and oxidative stress. Identification of BRCA1/2 mutation carriers is an essential component of cancer risk-reduction strategies and presents opportunities for cascade testing of other family members. Mutation carriers have several opportunities for cancer prevention or interception, including risk-reducing salpingo-oophorectomy or mastectomy, hormonal chemoprevention, and enhanced surveillance protocols, such as MRI-based breast cancer screening.

Prevention or early detection of BRCA1/2-related cancers is predicated on the identification of BRCA1/2 mutation carriers. At present, genetic testing for BRCA1/2 is only recommended for individuals with a known personal or familial history of breast, ovarian, tubal, or primary peritoneal cancer or for persons descending from populations with high mutational prevalence (e.g., Ashkenazi Jewish). However, more than half of all carriers with BRCA1/2 mutations have no family history of cancer, which would prompt a referral for genetic testing. Among the estimated 1 million BRCA1/2 mutation carriers in the United States, only 10% are aware of their carrier status.

With the cost of genetic testing making universal testing unfeasible, a functional screen for “BRCAness” could improve the efficiency of cancer early detection and prevention efforts, regardless of personal or family history. To this end, the present disclosure addresses the need for an effective method to stratify individuals as likely or unlikely to harbor a BRCA1/2 mutation through the use of microRNAs (miRNA) circulating in blood.

### BRIEF SUMMARY OF THE DISCLOSURE

One embodiment described herein is a method of predicting a lifetime risk of having one or more cancer, in a subject suspected of having cancer or a BRCA1 or BRCA2 mutation, including: (a) obtaining a sample collected from the subject; (b) determining the amounts of a circulating microRNA consisting of: hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19); (c) comparing the amounts of circulating microRNA as determined in step (b) to a statistical model; and (d) identifying the presence of at least one mutation in the BRCA1 or BRCA2 gene.

In one embodiment, step (b) includes determining the amount of ten microRNAs of hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), and hsa-miR-375-3p (SEQ ID NO: 10) in the sample.

In another embodiment, the cancer includes one or more of breast cancer, ovarian cancer, pancreatic, or prostate cancer.

In another embodiment, the sample is selected from a sample of blood. In another embodiment, the blood sample is selected from the group consisting of plasma, serum, and whole blood.

In another embodiment, the statistical model includes one or more models selected from the group consisting of linear discriminant analysis, logistic regression mode, multivariate adaptive regression splines, naïve Bayes, neural network, support vector machine, functional tree, LAD tree, Bayesian network, elastic net regression, and random forest. In one embodiment, the statistical model includes a logistic regression model. In another embodiment described herein, step (b) and/or step (d) are performed using RNA sequencing.

In another embodiment, the statistical model includes dimensionality reduction techniques. In another embodiment described herein, the joint lasso methods may be used for dimensionality reduction. In yet another embodiment, the model includes joint lasso dimensionality reduction and sparse machine learning techniques.

In another embodiment of the method, the method further includes performing genetic testing or genetic counseling.

In another embodiment of the method, the method further includes administering a treatment to the subject, wherein the treatment is selected from the group consisting of surgery, chemotherapy, immunotherapy, radiation therapy, hormone therapy, and stem cell transplant.

In one embodiment described herein, the subject is female.

Another embodiment described herein is a method of identifying a subject suspected of having a BRCA mutation, the method including: obtaining a sample collected from the subject; determining the amounts of a circulating microRNA consisting of: hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19); comparing the amounts of circulating microRNA as determined in step (b) to a statistical model; identifying the presence of at least one mutation in the BRCA1 or BRCA2 gene; and performing genetic testing on the subject. A subject suspected of having a BRCA mutation can refer to a subject at risk of having a BRCA mutation, regardless of any symptoms the subject may have.

In one embodiment, step (b) includes determining the amount of ten microRNAs of hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), and hsa-miR-375-3p (SEQ ID NO: 10) in the sample.

In another embodiment, the method further includes monitoring the subject for a BRCA associated cancer. In another embodiment, the BRCA associated cancer includes one or more of breast cancer, ovarian cancer, pancreatic, or prostate cancer.

In another embodiment, the method further includes administering a treatment to the subject, wherein the treatment is selected from the group consisting of surgery, chemotherapy, immunotherapy, radiation therapy, hormone therapy, and stem cell transplant.

In another embodiment described herein, is method of treating a patient suspected of having a BRCA associated cancer, the method including: (a) obtaining a sample collected from the subject; (b) determining the amounts of a circulating microRNA consisting of: hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19); (c) comparing the amounts of circulating microRNA as determined in step (b) to a statistical model; (d) identifying the presence of at least one mutation in the BRCA1 or BRCA2 gene; and (e) administering to the subject a treatment selected from the group consisting of surgery, chemotherapy, immunotherapy, radiation therapy, hormone therapy, and stem cell transplant.

In another embodiment is a kit including at least one test probe capable of specifically hybridizing to a microRNA selected from the group consisting of hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19).

In one embodiment of the kit, at least one of the probes includes a detectable label. In another embodiment, the kit includes a reagent for reverse transcription of a microRNA molecule.

In various embodiments, each of the foregoing embodiments can be used in combination with any of the other recited embodiments.

### BRIEF DESCRIPTION OF THE FIGURES

FIG. 1A is a table showing the six serum biorepositories used for the study group, based at: Brigham and Women's Hospital (BWH; Boston, MA; N=87), Dana-Farber Cancer Institute (DFCI; Boston, MA; N=200), including a separate sample set from the Center for Cancer Genetics and Prevention at DFCI (CCGP; Boston, MA; N=162), Tata Medical Center (DGO; Kolkata, India; N=20), Pomeranian Medical University (IHCC; Szczecin, Poland; N=52), and University of Pennsylvania (UPenn; Philadelphia, PA; N=132).

FIG. 1B is a flowchart showing the two versions of differential expression analysis performed.

FIG. 2A is a PCA representation of samples from all evaluated cohorts without batch adjustment.

FIG. 2B is a PCA representation of samples from all evaluated cohorts after batch adjustment with UPenn cohort as unmodified reference.

FIG. 3A is a PCA representation showing that BRCA status strongly affected expression profiles in the unadjusted batch shown in FIG. 2A.

FIG. 3B is a PCA representation of samples from all evaluated cohorts showing that BRCA status strongly affected expression profiles in the adjusted batch of FIG. 2B.

FIG. 3C is a scattergraph showing differentially expressed (DE) miRNAs according to germline BRCA1/2 mutations by superimposing the results after two strategies of data preprocessing—on raw data.

FIG. 3D is a scattergraph showing differentially expressed (DE) miRNAs according to germline BRCA1/2 mutations by superimposing the results after two strategies of data preprocessing—after batch adjustment.

FIG. 3E shows a heatmap of expression values of the miRNA the unsupervised hierarchical clustering of all subject samples from the 5 groups used for miRNA selection and model development showed that the samples clustered based on the BRCA1/2 mutations.

FIG. 4 shows a heatmap of expression values of miRNAs used in the classification model in the UPenn group.

FIG. 5A shows the ROC curve of the final logistic regression model the area under the curve equaled 0.89 (95% CI: 0.87-0.93).

FIG. 5B is a boxplot showing the predicted BRCA1/2 mutation probability in training, testing, and validation sets according to reference mutational status.

FIG. 5C is a boxplot showing the predicted BRCA1/2 mutation probability in the context of menopausal status.

FIG. 5D is a boxplot showing the predicted BRCA1/2 mutation probability in the context of having ovaries at the time of testing. FIG. 5C and FIG. 5D show that both menopausal status and lack of ovaries did not influence predicted BRCA1 or BRCA2 mutation probability. In boxplots median is marked as central line, boxes indicate the first and third quartile and whiskers present 1.5×IQR. FIGS. 5B, 5C and 5D present all N=653 samples, statistics were derived using all samples in respective subgroups.

FIG. 6A shows a ROC curve for BRCA1/2 classification using joint lasso.

FIG. 6B shows a confusion matrix using the maximum probability threshold t corresponding to the Youden's index, as highlighted on the ROC curve. In the confusion matrix, 0's correspond to negatives (e.g., non-BRCA), and 1's correspond to positives (e.g., BRCA).

FIG. 7A shows ROC comparisons of joint lasso model (blue, upper curves) and the metadata component of the joint lasso model (red, lower curves), i.e., using only metadata (e.g., BRCA family history) for model training, and the x axis component of the central figure in FIG. 6. This figure shows results using all subjects and a full data model.

FIG. 7B shows ROC comparisons of joint lasso model (blue, upper curves) and the metadata component of the joint lasso model (red, lower curves), i.e., using only metadata (e.g., BRCA family history) for model training, and the x axis component of the central figure in FIG. 6. This figure shows results using BRCA tested subjects and a full data model.

FIG. 7C shows ROC comparisons of joint lasso model (blue, upper curves) and the metadata component of the joint lasso model (red, lower curves), i.e., using only metadata (e.g., BRCA family history) for model training, and the x axis component of the central figure in FIG. 6. This figure shows results using all subjects and a limited data model.

FIG. 7D shows ROC comparisons of joint lasso model (blue, upper curves) and the metadata component of the joint lasso model (red, lower curves), i.e., using only metadata (e.g., BRCA family history) for model training, and the x axis component of the central figure in FIG. 6. This figure shows results using BRCA tested subjects and a limited data model.

FIG. 8A shows BRCA prediction results with 20 miRNA and 5 metadata features.

FIG. 8B shows the confusion matrix of BRCA prediction results with 20 miRNA and 5 metadata features.

FIG. 8C shows classification AUC with varying numbers of miRNA (1≤k₁≤20), with k₂=5.

FIG. 9A shows model performance on cancer history. Top row: ROC plot. Bottom row: sensitivity (TPR) and specificity (TNR) score among the subgroup using t=0.04 BRCA probability used in FIG. 6.

FIG. 9B shows model performance on age. Top row: ROC plot. Bottom row: sensitivity (TPR) and specificity (TNR) score among the subgroup using t=0.04 BRCA probability used in FIG. 6.

FIG. 9C shows model performance on race. Top row: ROC plot. Bottom row: sensitivity (TPR) and specificity (TNR) score among the subgroup using t=0.04 BRCA probability used in FIG. 6.

FIG. 10A shows model performance stratified on cancer history using the more limited set of 20 miRNA and 5 metadata features.

FIG. 10 B model performance stratified on age using the more limited set of 20 miRNA and 5 metadata features.

FIG. 10 C shows model performance stratified on race using the more limited set of 20 miRNA and 5 metadata features.

FIG. 11A shows BRCA score distribution after 10-fold cross validation, using both miRNA and metadata to train a joint lasso model.

FIG. 11B shows box plots of mean BRCA scores stratified by germline status and history of breast or ovarian cancer. In the box plot, the p values indicated whether there is a statistically significant difference in mean BRCA score. The calculations above show the mean and standard deviation values in the box plot and give the corresponding Fold Change (FC) and p values for each comparison. Here, the label “cancer” indicates a subject that has on record a previous diagnosis of ovarian or breast cancer.

FIG. 12A shows TSNE plots of miRNA and show the distribution of non-BRCA, BRCA1, and BRCA2 subjects. The plot shows significant linear separation between non-BRCA and BRCA (either 1 or 2) subjects in the miRNA data. There is little separation between BRCA1 and BRCA2.

FIG. 12B shows TSNE plots of miRNA and metadata and show the distribution of non-BRCA, BRCA1, and BRCA2 subjects. There is little separation between BRCA1 and BRCA2.

FIG. 13A shows external validation results in PLCO data with full data.

FIG. 13B shows external validation results in PLCO data with limited feature model results.

FIG. 13C shows external validation results in PLCO data with full data. The confusion matrix corresponding to the Youden indices in the training ROC curves from FIG. 13A is shown.

FIG. 13D shows external validation results in PLCO data with limited feature model results. The confusion matrix corresponding to the Youden indices in the training ROC curves from FIG. 13B is shown. Note: the relative risk curves become more noisy when BRCA score >0.7 due to small sample numbers (e.g., most PLCO subjects had BRCA scores <0.7).

FIG. 13E shows external validation results in PLCO with full data. The upward trend in relative cancer risk with BRCA score observed for FIG. 13E has R=0.80 (p<0.0001).

FIG. 13F shows external validation results in PLCO data with limited feature model results. For FIG. 13F, R=0.92 (p<0.0001). Note: the relative risk curves become more noisy when BRCA score >0.7 due to small sample numbers (e.g., most PLCO subjects had BRCA scores <0.7).

FIG. 14 shows a schematic of a classification procedure. The central figure shows the result of the joint lasso dimension reduction on the test samples after 10-fold cross validation. This is done for visualization, to show how the BRCA and non-BRCA samples separate. When the model is validated, only the training samples are used to train the classifier in the last step. x₁, x₂, and x₃ in the left-hand scatter plots denote the TSNE components.

### DETAILED DESCRIPTION

The present disclosure is directed to the methods of predicting lifetime risk of having one or more cancers, or of identifying a BRCA1 or BRCA2 mutation in a subject, and methods of identifying a subject suspected of having a BRCA mutation.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

It is to be understood that the methods described in this disclosure are not limited to particular methods and experimental conditions disclosed herein; as such methods and conditions may vary. It is also to be understood that the terminology used herein is for the purpose of describing particular embodiments only, and is not intended to be limiting.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2^{nd} edition).

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. The following references provide one of skill with a general definition of many of the terms used in the instant disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991). As used herein, the following terms have the meanings as ascribed to them below, unless specified otherwise.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, “administer” refers to the direct application of an agent to a subject. In embodiments, a medication is administered by ingestion, inhalation, infusion, injection, or any other means, whether self-administered or administered by a clinician or other medical professional.

As used herein, the term “nucleic acid” refers to a polymer of two or more nucleotides or nucleotide analogues (such as ribonucleic acid having methylene bridge between the 2′-O and 4′-C atoms of the ribose ring) capable of hybridizing to a complementary nucleic acid. As used herein, this term includes, without limitation, DNA, RNA, LNA, and PNA.

The term “microRNAs” or “miRNAs” as used herein, refers to small noncoding ribonucleic acid (RNA) gene products between 19 and 26 nucleotides long that form a hairpin secondary structure. MicroRNAs described herein are named using the nomenclature set forth in Ambros et al., RNA, 2003 Mar. 9(3):277-9, incorporated herein by reference, and sequences may be found at mirbase.org.

As used herein, the phrase “determining the amounts” refers to quantifying an analyte, such as a microRNA in a biological sample (e.g. a blood sample) using one or more detection techniques for detecting the analyte (such as qPCR, microarray detection, etc.) and quantifying using methods known in the art. An analyte that is detected in a biological sample using a detection technique is considered “present”. An analyte that is not detected in a biological sample using a detection technique is considered “absent”.

As used herein, the term “bind” or “binding” refers to non-covalent or covalent interaction between two molecules, such as between two complementary nucleic acids.

As used herein, the term “specifically hybridizing” refers to non-covalent interaction between a first nucleic acid molecule (e.g. a nucleic acid probe having a certain nucleotide sequence) and a second nucleic acid molecule (e.g. a microRNA having a nucleotide sequence complementary to that of the nucleic acid probe). Hybridization conditions have been described in the art and are known to one of skill in the art. In some embodiments, the condition for detecting the hybridization is a suitable condition of a nucleic acid detection assay (e.g., microarray, RT-PCR, or RT-qPCR). The likelihood of hybridization between two nucleic acids correlates with the nucleotide sequence complementary between the two nucleic acids.

The term “hybridize” as used herein, refers to annealing of a first single-stranded nucleic acid to a second complementary single-stranded nucleic, in which complementary nucleotides of the first and second nucleic acids pair by hydrogen bonding.

The phrase “detecting binding of a probe”, as used herein, refers to use of a detection method allowing determination that a probe (e.g. a nucleic acid probe) has non-covalently or covalently interacted with a target molecule (e.g. a target nucleic acid in a sample). For example, detecting binding of probe in qPCR may include optical detection of fluorescence of a self-quenching probe following binding to the complementary sequence of a target nucleic acid in the sample. In some embodiments, detecting binding of a probe may include detection of a nucleic acid intercalating agent to detect amplified double-stranded nucleic acid, such as a fluorescent intercalating agent used in qPCR.

As used herein, the term “probe” refers to a molecule or complex that is used to determine the presence or absence and/or amount of a microRNA in a sample (e.g. a blood sample). In certain embodiments, the probe includes a nucleic acid moiety (e.g., DNA, modified DNA, or modified RNA) that is capable of specifically hybridizing to the microRNA or a complementary DNA (cDNA) thereof. In certain embodiments, the probe includes a sequence of at least 15, 16, 17, 18, 19, 20, 21, 22, 23, 24, or 25 contiguous nucleotides identical or complementary to the microRNA. In certain embodiments, a probe further includes a detectable label that is conjugated, covalently or non-covalently, to the nucleic acid moiety. Exemplary detectable labels include without limitation a fluorophore, a small molecule (e.g., a small molecule of the avidin family), an enzyme, an antibody or antibody fragment, or a nucleic acid sequence not present in the subject in a form that is linked to the microRNA (e.g., a barcode sequence). Accordingly, the probe may be a fluorophore-labeled nucleic acid having a nucleotide sequence that is complementary to a nucleotide sequence of a microRNA.

The term “PCR”, as used herein, refers to polymerase chain reaction for amplifying an amount of target DNA. PCR relies on thermal cycling, which consists of cycles of repeated heating and cooling of a reaction for DNA denaturation, annealing and enzymatic elongation of the amplified DNA. First, the strands of the DNA are separated at a high temperature in a process called DNA melting or denaturing. Next, the temperature is lowered, allowing the primers and the strands of target DNA to selectively bind or anneal, creating templates for DNA polymerase to amplify the target DNA. Next, at a working temperature of the DNA polymerase, template-dependent DNA synthesis occurs. These steps are repeated to create many copies of the target DNA.

A “primer”, as used herein, refers to a short, single-stranded DNA sequence that selectively binds to a target DNA sequence and enables addition of new deoxyribonucleotides by DNA polymerase at the 3′ end. According to certain embodiments, the forward primer is 18-35, 19-32 or 21-31 nucleotides in length. The nucleotide sequence of the forward primer is not limited, so long as it specifically hybridizes with part of or an entire target site, and its melting temperature (“Tm”) value may be within a range of 50° C. to 72° C., in particular may be within a range of 58° C. to 61° C., and may be within a range of 59° C. to 60° C. The nucleotide sequence of the primer may be manually designed to confirm the Tm value using a primer Tm prediction tool. Primer nucleotides may include nucleotide analogues and/or modified nucleotides, such as LNA or PNA.

As used herein, the term “RT-PCR” refers to reverse transcription polymerase chain reaction, a process for amplifying RNA RNA molecules are reverse transcribed to complementary DNA (cDNA) using reverse transcriptase and then using PCR to amplify the resulting cDNA.

As used herein, the term “RT-qPCR” refers to reverse transcription quantitative polymerase chain reaction, a variant of RT-PCR in which amplification of cDNA during the RT-PCR process is quantitatively detected in real time using a probe that detects amplified target DNA. For example, in some embodiments, self-quenching nucleic acid probes are added to the reaction mixture. The self-quenching nucleic acid probes only fluoresce when they bind a target sequence. As each cycle of PCR is completed, the self-quenching probes bind to the amplified DNA, unquench and fluoresce with exposure to a light excitation source. As DNA is amplified, increased probe and target binding results in increased fluorescence of the self-quenching nucleic acid probe. Detection of the fluorescing probes after each amplification cycle allows real-time measurement of the amplification process, as increasing amounts of the nucleic acid probe bind with amplified target DNA and fluoresce. In some embodiments, an intercalating dye probe is added to the reaction mixture that fluoresces upon interaction with double-stranded nucleic acids. The increase in dye fluorescence during the amplification process allows the measurement of DNA amplification in real-time, as increasing amounts of the dye probe intercalate with the increasing amounts of target DNA being amplified.

As used herein, the term “normalize” or “normalizing” refers to adjusting a first measured value (e.g., level of a gene of interest) relative to a second measured value (e.g., level of a housekeeping gene), wherein the first and second measured values are measured from the same sample (e.g., different portions of the same homogenous sample), and wherein the second measured value is correlated to the quantity and/or quality of the sample. Normalization allows obtaining a relative amount of the first value that is not affected by the quantity and/or quality of the sample that may vary from individual sample preparation.

As used herein, the term “normalizing microRNA” refers to a microRNA that is known to have a stable amount in a sample (e.g. a blood sample) and is used to normalize the measured value of a test microRNA in the sample. A single normalizing microRNA may be used to normalize the measured amount of a target microRNA in a sample, or an averaged value of multiple microRNAs may be used for normalization. In certain embodiments, normalization may be calculated by: Number of amplification cycles (average of the normalizer microRNA)−number of amplification cycles (miRNA of interest).

As used herein, the term “test microRNA” refers to a microRNA the presence or absence and/or amount of which is determined, for example, for diagnosis purpose (e.g., using an algorithm). In some embodiments, the presence or absence and/or amount of one or more test microRNAs can be used additionally for normalization purpose.

As used herein, the term “normalizing probe” refers to a probe that is used to determine the presence or absence and/or amount of a normalizing microRNA in a sample (e.g. a blood sample). In certain embodiments, the normalizing probe includes a nucleic acid moiety (e.g., DNA, modified DNA, or modified RNA) that is capable of specifically hybridizing to a normalizing microRNA or a complementary DNA (cDNA) thereof.

As used herein, the term “test probe” refers to a probe that is used to determine the presence or absence and/or amount of a test microRNA in a sample (e.g. a blood sample). In certain embodiments, the test probe includes a nucleic acid moiety (e.g., DNA, modified DNA, or modified RNA) that is capable of specifically hybridizing to a test microRNA or a complementary DNA (cDNA) thereof.

The phrase “a reagent for amplification of a DNA sequence” includes, but is not limited to: (1) a heat-stable DNA polymerase; (2) deoxynucleotide triphosphates (dNTPs); (3) a buffer solution, providing a suitable chemical environment for optimum activity, binding kinetics, and stability of the DNA polymerase; (4) bivalent cations such as magnesium or manganese ions; and (5) monovalent cations, such as potassium ions. The reagents may be provided in the form of a solution, a concentrated solution, or powder.

The phrase “a reagent for reverse transcription of an RNA molecule” encompasses, but is not limited to: a reverse transcriptase; an RNase inhibitor; a primer that hybridizes to a nucleic acid sequence (such as RNA or DNA); a primer that hybridizes to an adenosine oligonucleotide; and a buffer solution that provides a suitable chemical environment for optimum activity, binding kinetics, and stability of the reverse transcriptase. The reagents may be provided in the form of a solution, a concentrated solution, or powder.

As used herein, the term “blood sample” refers to an amount of blood taken from a subject, such as whole blood, or a component portion of blood taken from a subject, such as plasma, which lacks cells normally contained in whole blood (e.g. erythrocytes, leukocytes, and platelets), or serum which is plasma that lacks fibrinogen and some clotting factors.

As used herein, the term “nucleic acid detection method” encompasses any method that may be used to detect the presence of a nucleic acid, including methods of sequencing (e.g. Gilbert sequencing, Sanger sequencing, SMRT sequencing or next-generation sequencing), microarray detection, PCR, RT-PCR, real-time qPCR, real-time RT-qPCR.

As used herein, the term “next-generation sequencing” refers to high-throughput parallel sequencing of short fragments of single-stranded nucleic acids attached to slides or beads, such as techniques by Illumina, Roche (454 sequencing), or Ion Torrent, Thermofisher. The incorporation of individual nucleotides onto single-stranded nucleic acids may be detected optically (via fluorescence of incorporated nucleotides) or by detection of hydrogen ions released during nucleotide incorporation (e.g., ion semiconductor sequencing).

As used herein, the term “artificial neural network” refers to a forecasting model based on a linked collection of neural units in silico that loosely model a simple mathematical model of the brain. Artificial neural networks allow identification of complex nonlinear relationships between its response variable and its predictor variables. An artificial neural network may have one or more hidden layers that each include one or more neurons that interact to produce a prediction given two or more variables.

As used herein, the term “cancer” relates generally to a class of diseases or conditions in which abnormal cells divide without control and may invade nearby tissues.

As used herein, the term “cancerous cell,” “cancer cell,” “tumor cell” or variant thereof refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, do not necessarily form tumors. For those cancers that form tumors, the terms cancer (cell) and tumor (cell) are used interchangeably. The amount of a tumor in an individual is the “tumor burden” which may be measured as the number, volume, or weight of the tumor.

As used herein the term “BRCA associated cancer” refers to a cancer associated with mutations in the BRCA genes that make cells more likely to divide and change rapidly, causing cancer.

The term “breast cancer”, as used herein, refers to a group of malignancies affecting breast tissue. Molecular classification of breast cancer has identified specific subtypes, often called “intrinsic” subtypes, with clinical and biological implications, including an intrinsic luminal subtype, an intrinsic HER2-enriched subtype (also referred to as the HER2+ or ER−/HER2+ subtype) and an intrinsic basal-like breast cancer (BLBC) subtype. All forms of breast cancer are contemplated herein. Approximately 45%-72% of women who inherit a BRCA1 or BRCA2 variant will develop breast cancer by age 70 to 80 years.

The term “ovarian cancer,” as used herein, refers to a group of malignancies affecting the ovary, that have developed from epithelial cells, sex cord-stromal cells (e.g. granulosa, theca, and hilus cells), or germ cells (e.g. oocytes). About 60% of ovarian tumors are of epithelial origin and account for 90% of ovarian cancers. Such epithelium-derived ovarian carcinomas are heterogeneous in character, with differences in tumor morphology, clinical symptoms, and genetic alterations. The World Health Organization (WHO) lists eight different tumor histologies, including serous, endometrioid, mucinous, clear cell, transitional cell, squamous cell, mixed epithelial, and undifferentiated. Tumors of each of these subtypes may be classified as benign (having low malignant potential and/or indolence), malignant, or borderline, as well as low-grade (Type I) or high-grade (Type II). All forms of ovarian cancer are contemplated herein.

As used herein the term “pancreatic cancer” refers to cancers that begin in the pancreas and is associated with poor prognosis and low survival rate. Treatment of pancreatic cancer includes surgery, chemotherapy, radiation therapy, and palliative care. The treatment options may depend on the stage of pancreatic cancer. BRCA1 and BRCA2 mutations are associated with familiar pancreatic cancer.

As used herein the term “prostate cancer” refers to the cancer of the prostate gland. Prostate cancer is a commonly diagnosed malignancy among men and the second leading cause of male cancer deaths in the western population, following lung cancer. If discovered, early prostate cancer may be cured with surgery in approximately 90% of cases. However, the disease is slowly fatal once the tumor spreads outside the area of the gland and forms distant metastases. Early detection and accurate staging are therefore of great importance for the accurate choice of therapy and should improve the success rate of treatments and reduce the mortality rate associated with prostate cancer. Up to 10% of all prostate cancers may be linked to inherited BRCA gene mutations.

As used herein, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant to include, and be limited to, whatever follows the phrase “consisting of.” Thus, the phrase “consisting of” indicates that the listed elements are required or mandatory, and that no other elements may be present. By “consisting essentially of” is meant including any elements listed after the phrase, and be limited to other elements that do not interfere with or contribute to the activity or action specified in the disclosure for the listed elements.

As used herein, the term “malignant” refers to a cancer in which a group of tumor cells display one or more of uncontrolled growth (e.g., division beyond normal limits), invasion (e.g., intrusion on and destruction of adjacent tissues), and metastasis (e.g., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primacy) tumor. As used herein, the term “benign” or “non-malignant” refers to tumors that may grow larger but do not spread to other parts of the body. Benign tumors are self-limited and typically do not invade or metastasize.

As used herein, the term “treating” or “treatment” refers to relieving, reducing, or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease, or prevention of at least one symptom associated with or caused by the state, disease or disorder being prevented. For example, treatments may relieve, reduce or alleviate at least one symptom of a BRCA associated cancer, e.g., breast cancer, ovarian cancer, pancreatic cancer or prostate cancer. Exemplary treatments of such cancers include, immunotherapy, pharmaceutical therapies, chemotherapy, and/or surgical procedures.

As used herein, “immunotherapy” refers to the prevention, amelioration of, or treatment of disease with substances that may stimulate an immune response. Examples of immunotherapies include but are not limited to, monoclonal antibodies or immune checkpoint inhibitors, non-specific immunotherapies, oncolytic virus therapy, T-cell therapy, cancer vaccines. As used herein “pharmaceutical therapies” refer to the administration of any small molecule chemical compound or composition thereof for the specific type of cancer or associated symptoms. For example, one class of pharmaceutical therapies are anti-inflammatory agents or drugs. Exemplary anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Chemotherapies include but are not limited to platinum-based chemotherapy (e.g., cisplatin or carboplatin and a taxane). In some instances, other chemotherapeutic agents may be used if the cancer is resistant to platinum-based drugs either alone or in combination, such as liposomal doxorubicin, paclitaxel, docetaxel, nab-paclitaxel, gemcitabine, etoposide, pemetrexed, cyclophosphamide, topotecan, vinorelbine, irinotecan, or PARP inhibitors. Surgical treatments may include, but are not limited to cytoreductive surgery (e.g., debulking to remove the tumor, salpingo-oophorectomy, hysterectomy, mastectomy, pancreaticoduodenectomy, or radical prostatectomy), followed by chemotherapy. Any appropriate treatment for the specific type of cancer is contemplated herein, including any appropriate combination of treatment, at the appropriate dosages and using the appropriate regimens, as prescribed by a physician.

Described herein is a method of using circulating miRNA, collectively having a specific signature or profile, for identifying individuals suspected of having a BRCA associated cancer or having one or more BRCA mutations. The method may also be used for any individual at risk of having a BRCA associated cancer or having one or more BRCA mutations. The methods described herein offer a simple, preliminary screening option, prior to genetic counseling or genetic testing for patients, with a known family history of BRCA1 or BRCA2 mutations and related cancers. Application of an miRNA-based test to identify patients at highest risk for these mutations offers an opportunity to reduce the costs of screening and make it widely available, but also to identify patients that are carrier of BRCA1 or BRCA2 mutations before the onset of cancers, such that individual may seek preemptive treatments or surgical interventions.

Thus, there is provided a method of predicting a lifetime risk of having one or more cancers, in a subject suspected of having BRCA mutation, including: (a) obtaining a sample collected from the subject; (b) determining the amounts of one or more of the miRNA sequences of a panel of circulating microRNA; (c) comparing the amounts of circulating microRNA as determined in step (b) to a statistical model; and (d) identifying the presence of at least one mutation in the BRCA1 or BRCA2 gene.

The well-established dysregulation of miRNA expression in cancer, together with the contribution of miRNAs to tumorigenesis, and the fact that in BRCA1/2 mutation carriers have genetic alterations present in all body cells, offers a distinct circulating miRNA signature or profile. In one embodiment the miRNA profile includes one or more miRNA sequences from Table 1:

TABLE 1
miRNA sequences that may be used in an miRNA profile.

| Sequence ID | | |
|---|---|---|
| No | Sequence Name | Sequence |
| 1 | hsa-let-7b-5p | UGAGGUAGUAGGUUGUGUGGUU |
| 2 | hsa-miR-17-5p | CAAAGUGCUUACAGUGCAGGUAG |
| 3 | hsa-miR-19b-3p | UGUGCAAAUCCAUGCAAAACUGA |
| 4 | hsa-miR-20b-5p | CAAAGUGCUCAUAGUGCAGGUAG |
| 5 | hsa-miR-30d-5p | UGUAAACAUCCCCGACUGGAAG |
| 6 | hsa-miR-139-3p | UGGAGACGCGGCCCUGUUGGAGU |
| 7 | hsa-miR-182-5p | UUUGGCAAUGGUAGAACUCACACU |
| 8 | hsa-miR-320b | AAAAGCUGGGUUGAGAGGGCAA |
| 9 | hsa-miR-421 | AUCAACAGACAUUAAUUGGGCGC |
| 10 | hsa-miR-375-3p | UUUGUUCGUUCGGCUCGCGUGA |
| 11 | hsa-miR-106b-5p | UAAAGUGCUGACAGUGCAGAU |
| 12 | hsa-miR-134-5p | UGUGACUGGUUGACCAGAGGGG |
| 13 | hsa-miR-493-5p | UUGUACAUGGUAGGCUUUCAUU |
| 14 | hsa-miR-500a-3p | AUGCACCUGGGCAAGGAUUCUG |
| 15 | hsa-miR-1273h-3p | CUGCAGACUCGACCUCCCAGGC |
| 16 | hsa-miR-4433a-3p | ACAGGAGUGGGGGUGGGACAU |
| 17 | hsa-miR-4433b-5p | AUGUCCCACCCCCACUCCUGU |
| 18 | hsa-miR-485-3p | GUCAUACACGGCUCUCCUCUCU |
| 19 | hsa-miR-1304-3p | UCUCACUGUAGCCUCGAACCCC |

In another embodiment the miRNA profile includes one or more miRNA sequences from Table 2:

TABLE 2
miRNA sequences that may be used in an miRNA profile.

| Sequence ID | | |
|---|---|---|
| No | Sequence Name | Sequence |
| 1 | hsa-let-7b-5p | UGAGGUAGUAGGUUGUGUGGUU |
| 2 | hsa-miR-17-5p | CAAAGUGCUUACAGUGCAGGUAG |
| 3 | hsa-miR-19b-3p | UGUGCAAAUCCAUGCAAAACUGA |
| 4 | hsa-miR-20b-5p | CAAAGUGCUCAUAGUGCAGGUAG |
| 5 | hsa-miR-30d-5p | UGUAAACAUCCCCGACUGGAAG |
| 6 | hsa-miR-139-3p | UGGAGACGCGGCCCUGUUGGAGU |
| 7 | hsa-miR-182-5p | UUUGGCAAUGGUAGAACUCACACU |
| 8 | hsa-miR-320b | AAAAGCUGGGUUGAGAGGGCAA |
| 9 | hsa-miR-421 | AUCAACAGACAUUAAUUGGGCGC |
| 10 | hsa-miR-375-3p | UUUGUUCGUUCGGCUCGCGUGA |

The miRNA profiles described herein are informative of BRCA1 or BRCA2 mutations and consistently demonstrate the ability to separate BRCA mutations from wild-type. Thus, the presence or absence of one or more of the miRNA sequences on the panel, and their subsequent quantification may identify patients that are carriers of this mutation, and be used to predict the lifetime risk of having one or more BRCA associated cancers. BRCA mutations may occur in both men and women and be inherited and several cancers are associated with BRCA1 or BRCA1. For example, BRCA1 mutations are associated with an increased risk of breast cancer, including triple-negative breast cancer. BRCA2 is associated with higher risk of other cancers including prostate and pancreatic cancer. Thus, in one embodiment, the cancers described herein comprise one or more of breast cancer, ovarian cancer, pancreatic, or prostate cancer.

MicroRNAs are endogenous non-coding small RNA molecules that can be secreted into the circulation and exist in remarkably stable forms. Thus, the use of circulating miRNAs is ideal for patient samples using blood that may be routinely drawn by a physician or clinic. In one embodiment, blood samples used with the methods described herein include plasma, serum or whole blood. Any appropriate blood sample may be used from which circulating miRNA's may be extracted, detected and/or quantified or measured. MicroRNA may be detected and quantified in a biological sample (e.g., a blood sample) using one or more detection techniques for detecting the analyte (such as qPCR, microarray detection, etc.) and quantifying using methods known in the art.

Once samples are obtained, the amounts of circulating miRNA are compared to a statistical model to differentiate between samples containing a BRCA mutation and wild-type samples. In one embodiment the statistical model employed as described herein includes one or more models selected from the group consisting of linear discriminant analysis, logistic regression mode, multivariate adaptive regression splines, naïve Bayes, neural network, support vector machine, functional tree, LAD tree, Bayesian network, elastic net regression, and random forest. In another embodiment, the statistical model includes a logistic regression model with the parameters shown in Table 3.

TABLE 3
Logistic regression model parameters used for predicting
BRCA status. Estimates, odds ratios, and two-sided p values
were estimated on the training set of samples. The final model
with a cut off value of 50% probability for declaring
positive cells was evaluated on the validation cohort.

| miRNA | Univariable analysis | | | | |
|---|---|---|---|---|---|
| miRNA | OR | (−95% CI) | (+95% CI) | p value | |
| Intercept | | | | | |
| hsa.miR.20b.5p | 0.26 | 0.20 | 0.33 | <0.001 | |
| hsa.miR.19b.3p | 0.19 | 0.14 | 0.26 | <0.001 | |
| hsa.let.7b.5p | 0.11 | 0.07 | 0.16 | <0.001 | |
| hsa.miR.320b | 3.81 | 2.90 | 5.11 | <0.001 | |
| hsa.miR.139.3p | 3.16 | 2.46 | 4.11 | <0.001 | |
| hsa.miR.30d.5p | 0.19 | 0.13 | 0.27 | <0.001 | |
| hsa.miR.17.5p | 0.32 | 0.25 | 0.41 | <0.001 | |
| hsa.miR.182.5p | 0.37 | 0.30 | 0.46 | <0.001 | |
| hsa.miR.421 | 0.37 | 0.28 | 0.49 | <0.001 | |
| hsa.miR.375.3p | 1.64 | 1.44 | 1.87 | <0.001 | |
| | Multivariable analysis | | | | |
| | Estimate | OR | (−95% CI) | (+95% CI) | p value |
| Intercept | 34.23 | | | | |
| hsa.miR.20b.5p | −0.21 | 0.81 | 0.55 | 1.17 | 0.300 |
| hsa.miR.19b.3p | −0.81 | 0.44 | 0.27 | 0.71 | <0.001 |
| hsa.let.76.5p | −2.05 | 0.13 | 0.07 | 0.24 | <0.001 |
| hsa.miR.320b | 0.94 | 2.55 | 1.70 | 3.92 | <0.001 |
| hsa.miR.139.3p | 0.23 | 1.26 | 0.87 | 1.82 | 0.200 |
| hsa.miR.30d.5p | −0.90 | 0.41 | 0.24 | 0.67 | <0.001 |
| hsa.miR.17.5p | −0.04 | 0.96 | 0.63 | 1.44 | 0.800 |
| hsa.miR.182.5p | 0.39 | 1.48 | 1.03 | 2.16 | 0.036 |
| hsa.miR.421 | 0.30 | 1.36 | 0.95 | 1.95 | 0.093 |
| hsa.miR.375.3p | 0.27 | 1.31 | 1.09 | 1.58 | 0.004 |

The ability to identify patients having a BRCA1 mutation, BRCA2 mutation, both, or the ability to predict the lifetime risk of subjects having one or more cancers allows the opportunity for continued screening and potential treatment interventions if cancer is present. Treatment options include all known treatment options for any of the cancers described herein, including immunotherapy, pharmaceutical therapy, chemotherapy, surgical interventions and combinations thereof. In one embodiment, the methods described herein further include administering a treatment to the subject, wherein the treatment is selected from the group consisting of surgery, chemotherapy, immunotherapy, radiation therapy, hormone therapy, and stem cell transplant. Treatments may be prescribed and dosed as recommended by a physician.

The instant disclosure further contemplates kits containing the reagents for the detection of miRNA molecule in the methods as described herein. In some embodiments, the kits may further include instructions for use in accordance with the methods of this disclosure, including instructions for how to draw blood, treat a sample, and perform the detection of a BRCA1 or BRCA2 mutation. The kit may further include instructions for generating a risk score of developing a BRCA associated cancer based on the results of the detection. Furthermore, the kit may include references for follow up genetic screening and/or genetic counseling, and or follow up diagnostics screening patients for potential cancer.

The practice of the present disclosure offers numerous improvements and advantages over other techniques. The methods described herein include serum analyte (miRNA) and clinical metadata (personal and family history) to improve screening tests for BRCAness, or BRCA1/2 mutations. BRCAness refers to a surrogate biomarker for cancer risk, such as near-term ovarian cancer risk. The techniques described herein perform consistently well across numerous categories, including age, type of cancer, and race. Additionally, the methods described herein have unique applications for forecasting ovarian cancer risk, including determining a 5-year risk of ovarian cancer. The methods described herein can be used as a “screening test.” In other words, subjects can be tested for miRNA and clinical metadata to determine if it is advantageous to refer the subject for further genetic testing. This improves the cost and time required to test for BRCA1/2 mutations.

The model's performance remained constant throughout the whole range of age categories. The model incorporates clinical metadata and a miRNA assessment of BRCAness.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

The following examples are put forth so as to provide those of ordinary skill in the art with a complete disclosure and description of how to make and use the methods and panel described herein, and are not intended to limit the scope of the instant disclosure.

### EXAMPLES

### General Methodology

The following examples are included for illustrative purposes only and are not intended to limit the scope of the invention.

### Example 1

The aim of this study was to develop a serum-based miRNA panel to identify BRCA1/2 mutation carriers among healthy controls. A diagnostic biomarker study was performed based on serum samples collected between by six international cohorts (BWH—Brigham and Women's Hospital; CCGP—Center for Cancer Genetics and Prevention at DFCI; DGO—Department of Gynaecological Oncology, Tata Medical Center, Kolkata, India; IHCC—International Hereditary Cancer Center of the Pomeranian Medical University, Poland; DFCI—DFCI/BWH biobank; UPenn—University of Pennsylvania). Serum samples from 653 healthy participants with known mutation status of BRCA1 and BRCA2 were used in the analysis. Area under the receiver operating characteristic curve (AUC ROC), sensitivity and specificity (assessed in an independent validation set of samples) were used to assess the performance of the final classification model. Among the study population, 350 (53.6%) subjects had BRCA mutations and 303 (46.4%) were BRCA1/2—wild-type. In all individuals, miRNAs were isolated and expression quantified using RNA sequencing. Variable selection based on differential expression analysis on merged, batch adjusted cohorts, identified a panel of 19 miRNAs significantly associated with BRCA mutation carrier status, with 10 of them ultimately used for class separation through a logistic regression model.

The model achieved AUC ROC 0.89 (95% CI: 0.87-0.93) and 85.61% accuracy, 93.88% sensitivity and 80.72% specificity in the validation group. Mutation of either BRCA1 or BRCA2, menopausal status or having preemptive oophorectomy before blood sample draw did not affect classification performance. Circulating microRNAs may thus be used to identify BRCA1 or BRCA2 mutations in patients of high genetic risk of ovarian and breast cancer, facilitating cheap first line screening for further genetics studies.

### Samples

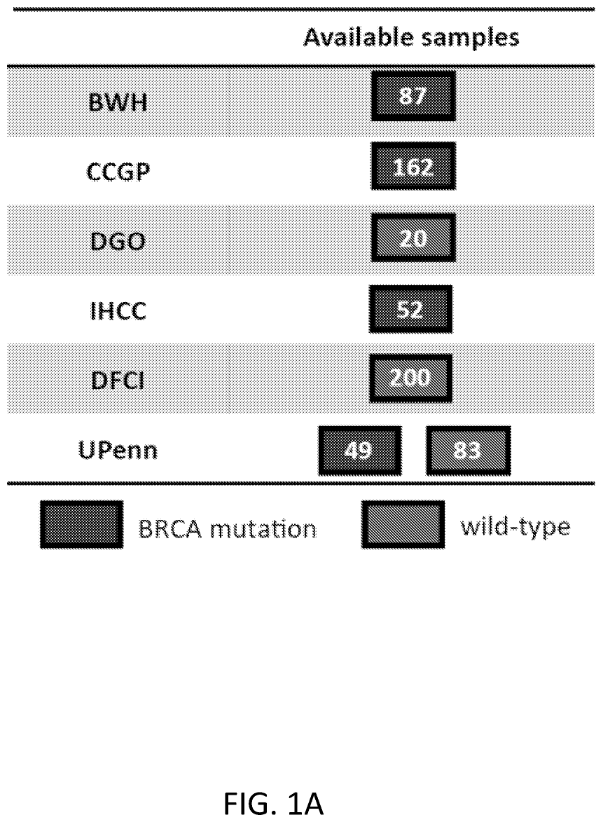

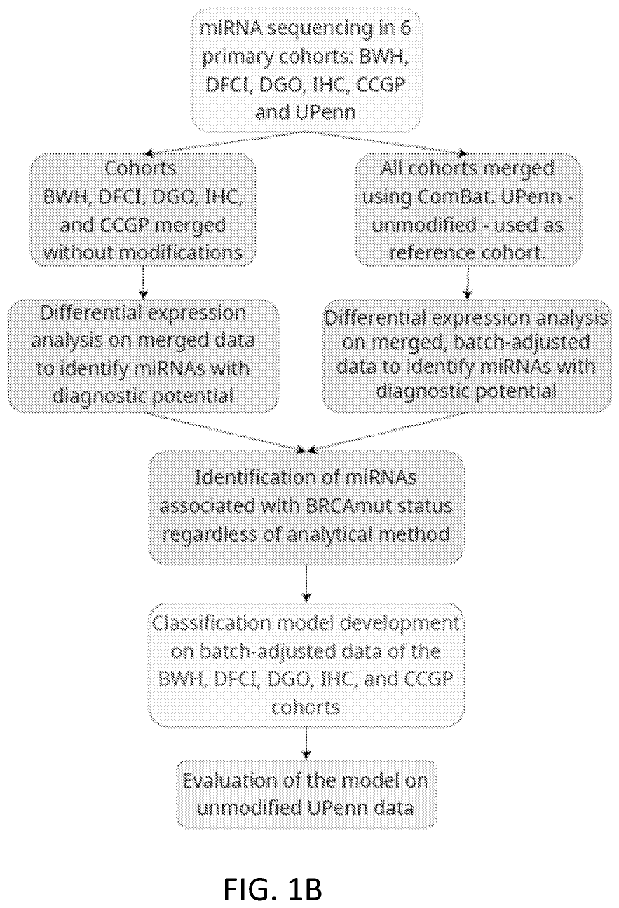

The study group was assembled from six serum biorepositories (FIG. 1A) based at: Brigham and Women's Hospital (BWH; Boston, MA; N=87), Dana-Farber Cancer Institute (DFCI; Boston, MA; N=200), including a separate sample set from the Center for Cancer Genetics and Prevention at DFCI (CCGP; Boston, MA; N=162), Tata Medical Center (DGO; Kolkata, India; N=20), Pomeranian Medical University (IHCC; Szczecin, Poland; N=52), and University of Pennsylvania (UPenn; Philadelphia, PA; N=132). Samples from patients with genetically-confirmed BRCA1/2 status were included in the study. Patients with ovarian cancer history or other cancer diagnosed within 1 year from sampling were excluded. Patients with benign adnexal masses were included. Patients with missing diagnoses or BRCA status were excluded. All study samples were collected under locally approved institutional review board protocols after obtaining informed consent from study subjects.

### Next Generation Sequencing

Total RNA was extracted, followed by size-selection, adaptor ligation, and library preparation as previously described. All miRNA sequencing data were mapped to the reference miRNA database (miRBase version 22.1) using nf-core/smrnaseq version 1.1.0, a uniform, standardized bioinformatic pipeline developed and published as a part of the Nextflow project. Reads unmapped to miRbase were subsequently mapped to human genome GRCh38. The sequencing protocol was set as QIAseq, Illumina® or Nextflex, as appropriate to each sample set (QIAseq miRNA sequencing in BWH, IHCC, DFCI and UPenn; Illumina miRNA sequencing in CCGP and NEXTFLEX small RNA sequencing in DGO). All parameters of the pipeline were kept at the default values recommended by the code authors to assure reproducibility.

Data Integration and miRNA Selection

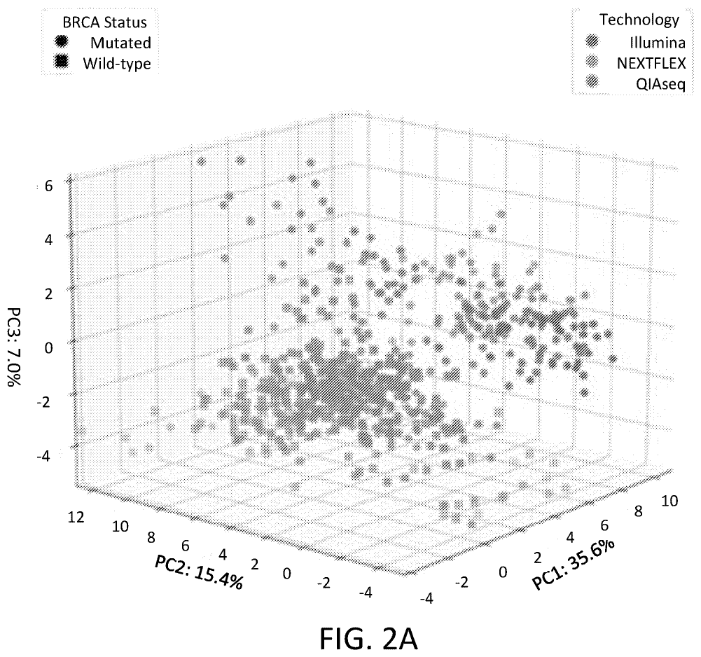

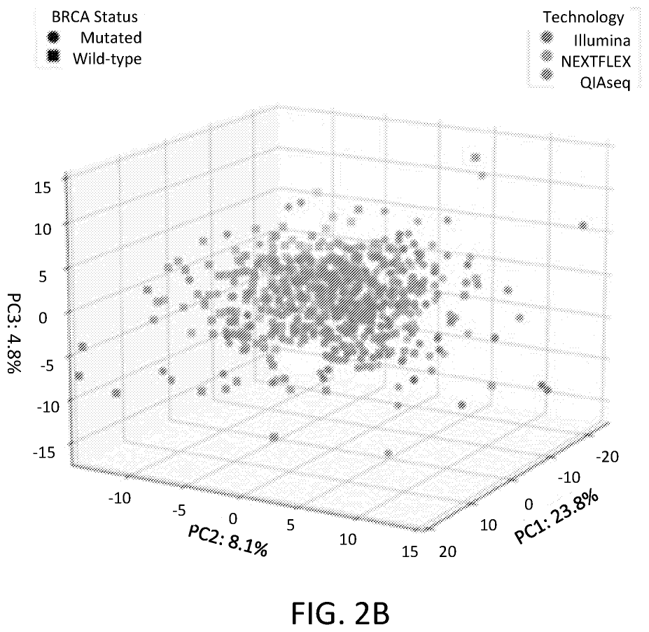

MicroRNAs were filtered for species detected in at least 33% of the samples in each group at a minimum detection threshold of >=10 transcripts per million (TPM). After filtering, 227 of initial 2621 miRNAs were retained. Principal Component Analysis (PCA) was used to visualize the presence of batch effects (FIG. 2). After voom normalization and mean variance trend removal (model formula used for voom: ~0+BRCAStatus+having Ovaries+group), ComBat was used to combine data from all subject groups, with the UPenn group serving as reference (model formula used for ComBat: ~brcaStatus+having Ovaries). As different technologies were used to quantify the miRNA content in different subject groups, use of an empirical Bayes framework (ComBat) was a necessary step to combine data from all subject groups while accounting for technical heterogeneity. However, to limit the potential confounding influence of ComBat on the effect of interest, two versions of differential expression analysis are performed (FIG. 1B): with and without batch adjustment, and compared the results to identify miRNAs detected in both variants. Differential expression analysis was performed using the limma linear model for microarray and RNA-seq data (bioconductor.org/packages/devel/bioc/vignettes/limma/inst/doc/usersguide.pdf). The model formula for limma included the following effects: BRCA1/2 mutation and the effect of prior bilateral salpingo-oophorectomy (~0+brcaStatus+having ovaries). Visualization of the samples in reduced dimensionality space was performed using uniform manifold approximation projection (UMAP). Settings were as follows: number of neighbors for representation: 10 for batch-adjusted data and 5 for unadjusted, minimal distance: 0.2 for batch-adjusted data and 0.9 for unadjusted, distance metric: Euclidean in both cases. Hierarchical clustering was performed using the Ward method for linkage, Euclidean distance metric for samples (columns) and correlation distance metric for miRNAs (rows).

### Model Development and Statistical Analysis

In this step, the dataset was divided into training (N=391, 75% of cases from all groups except UPenn, random split), testing (N=130, 25% of cases from all groups except UPenn, random split) and validation (N=132, only UPenn group) sets. Model development and validation was conducted using in-house OmicSelector software (version 1.0; biostat.umed.pl/OmicSelector). Briefly, OmicSelector tests 94 feature selection approaches based on 25 distinct variable selection methods. OmicSelector-based feature selection followed initial consistency-based preselection as described above. Feature sets with more than 10 miRNAs were filtered out. Selected feature sets were ranked using 4 modeling techniques (logistic regression, conditional decision trees, recursive partitioning trees and artificial neural networks with 1 hidden layer) with hyper-parameter optimization (2000 random hyper-parameter sets) and hold-out validation on the testing set. The number of modeling techniques were reduced to assure low complexity of resulting models, and thus reduce the chance of overfitting.

To assess model performance, training area under the ROC was analyzed and a cut-off value for BRCA status prediction was chosen based on the highest Youden index. This cut-off was applied for prediction on testing and validation sets. Accuracy, sensitivity, specificity, positive predictive value (PPV) and negative predictive value (NPV) were calculated for all sets. Where indicated, the alpha level for statistical significance was set at <0.05.

BRCA status and other available clinical data were known to researchers responsible for feature selection and models development. Models were developed with the use of results of genetic testing for BRCA status; thus, modeling outcomes were unknown when those tests were performed. All hypothesis tests were 2-sided. All analyses were performed in R (cran.r-project.org).

### Characteristics of the Study Population

The study population characteristics are summarized in Table 4.

TABLE 4
Clinical characteristics of the studied group used in Example 1.

| Variable | | Subjects (n = 635) | |
|---|---|---|---|
| Menopausal Status | Post-menopausal | 154 | (23.6%) |
| | Pre-menopausal | 187 | (28.6%) |
| | No data | 312 | (47.8%) |
| Age, median (IQR^{a}), years | | 49 | (39.2-60) |
| | | Unknown 212 | (47.6%) |
| CA-125, median (IQR), IU/ | | 15 | (10.3-31.2) |
| | | Unknown 552 | (84.5%) |
| Having ovaries at testing | Yes | 578 | (88.5%) |
| | No | 75 | (11.5%) |

^{a}IQR—interquartile range

In total, samples were collected from 653 study subjects. Among the study population, 350 (53.6%) subjects had BRCA mutations (BRCA-mt) and 303 (46.4%) were BRCA1/2—wild-type (BRCA-wt). Summary clinical characteristics of each group are presented in Table 5.

TABLE 5
Clinical characteristics of all cohorts used in Example 1.

| | | BWH | CCGP | DGO | IHCC | DFCI |
|---|---|---|---|---|---|---|
| Variable | Level | (N = 87) | (N = 162) | (N = 20) | (N = 52) | (N = 200) |
| Menopausal | Yes | 0 | 0 | 4 (20.0%) | — | 119 (59.5%) |
| Status | No | 0 | 0 | 5 (25.0%) | — | 81 (40.5%) |
| | Unknown | 87 (100%) | 162 (100%) | 11 (55.0%) | 52 (100%) | 0 |
| Age (years) | Median | — | — | 40.5 | — | 54.0 |
| | (25%-75%), | | | (29.0-58.8), | | (46.0-65.0), |
| | Min-Max | | | 28.0-71.0 | | 24.0-83.0 |
| | No data | 87 (100%) | 162 (100%) | 12 (60%) | 52 (100%) | 0 |
| Ca-125 | Median | — | — | 173.6 | — | 15.0 |
| | (25%-75%), | | | (21.7-328.4), | | (11.0-31.0), |
| | Min-Max | | | 8.6-2889.0 | | 0.0-2427.0 |
| | No data | 87 (100%) | 162 (100%) | 14 (70%) | 52 (100%) | 118 (59.0%) |
| Having | Yes | 40 (46%) | 134 (82.7%) | 20 (100%) | 52 (100%) | 200 (100%) |
| ovaries at | No | 47 (54%) | 28 (17.3%) | 0 | 0 | 0 |
| testing | | | | | | |

A small number of participants (75/653; 11.5%) had undergone risk-reducing salpingo-oophorectomy prior to blood collection because of BRCA-associated cancer risk which was accounted for in the differential expression analysis.

Identification of miRNAs Associated with Germline BRCA Mutations

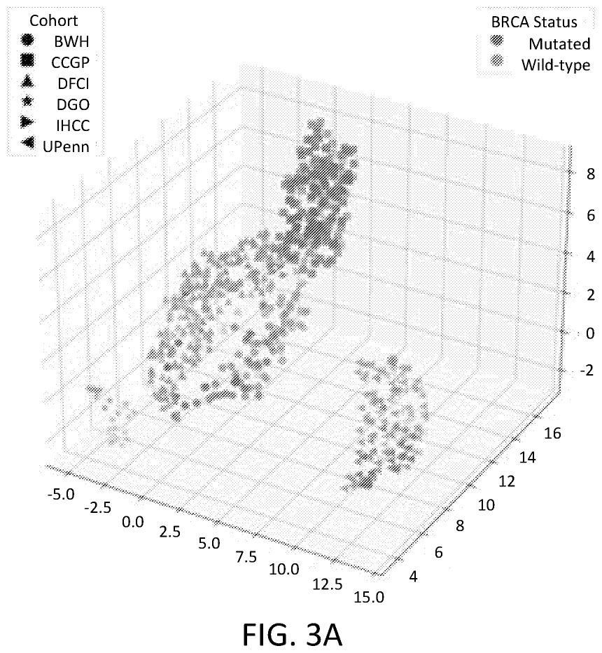

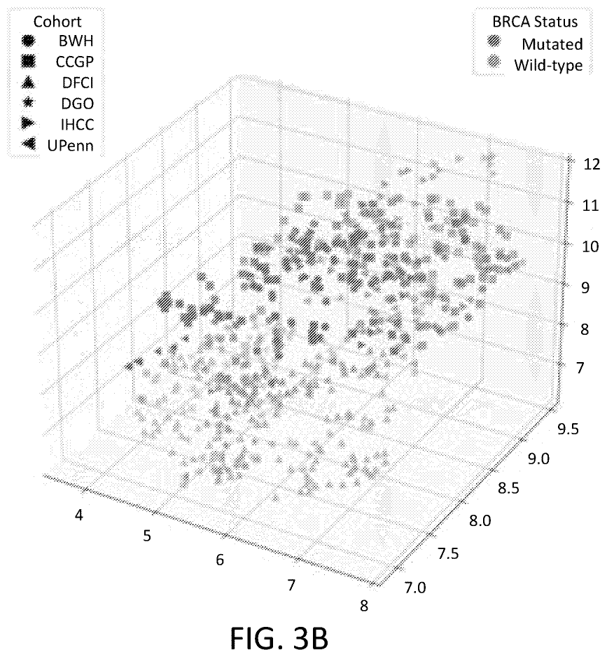

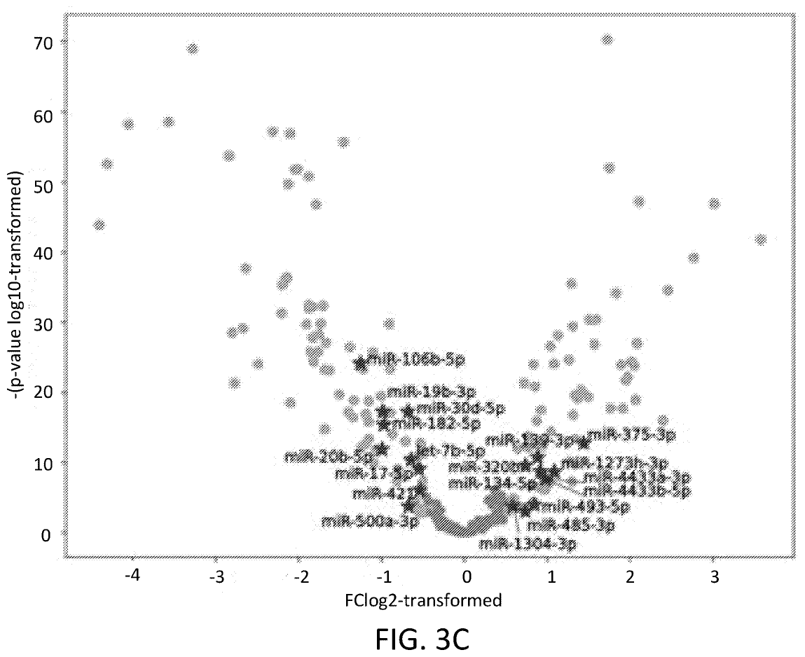

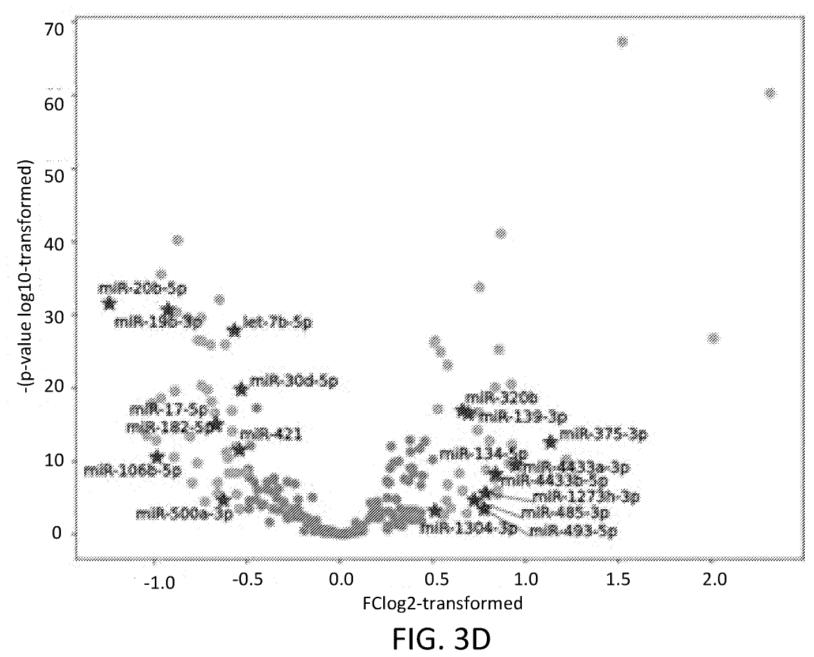

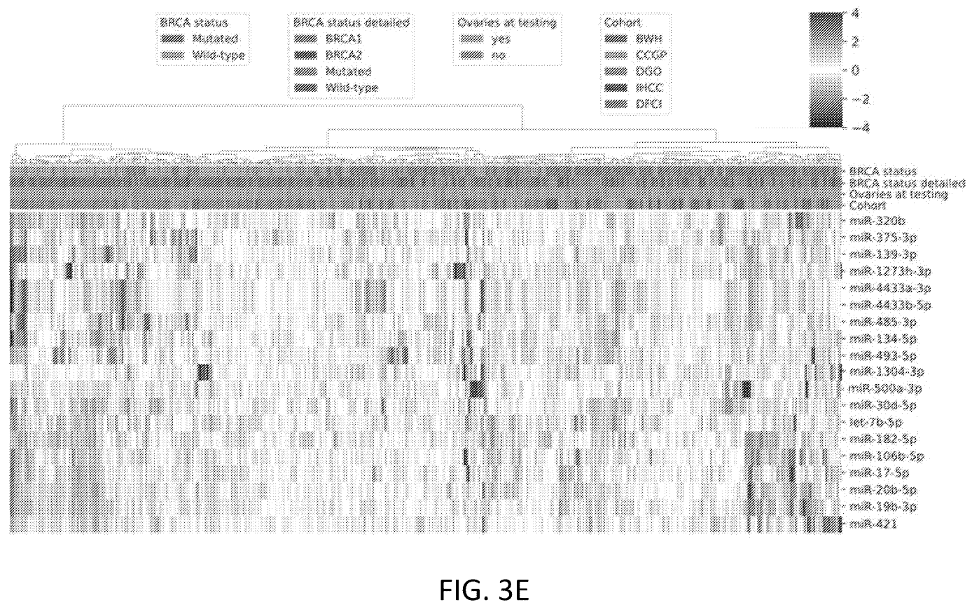

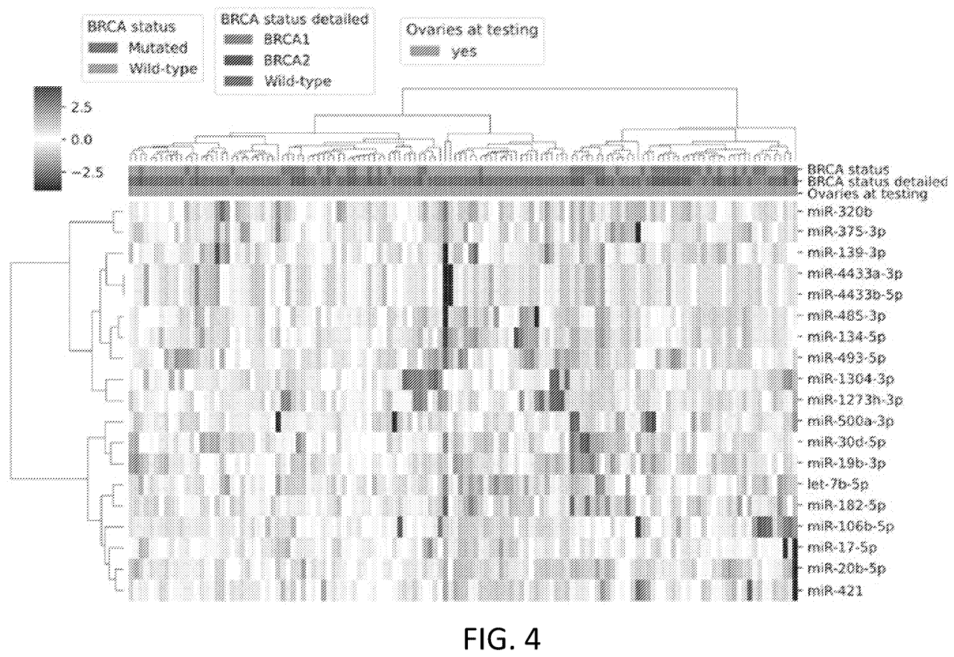

Unsupervised, linear and non-linear dimensionality reduction with PCA and UMAP were used to examine the effects of BRCA deficient status and that of the batch (FIG. 2). The batch effect clearly separated the groups. However, within both observed batches, the BRCA status strongly affected expression profiles (FIG. 3A). The goal was to identify differentially expressed (DE) miRNAs according to germline BRCA1/2 mutations by superimposing the results after two strategies of data preprocessing: on raw data (FIG. 3C) and after batch adjustment (FIG. 3D). Nineteen miRNAs were convergent (p<0.01 with |log 2(FC)|>0.5 in the same direction in both variants, ratio of FCs from two analysis variants between 0.8 and 1.25) regardless of the data preprocessing strategy (gray markings in FIG. 3C and FIG. 3D). Unsupervised hierarchical clustering of all subject samples from the 5 groups used for miRNA selection and model development showed that the samples clustered based on the BRCA1/2 mutations (FIG. 3E). Notably, in the validation group composed of UPenn samples, the 19 miRNAs also clearly separated BRCA-mt and BRCA-wt samples confirming the robustness of their selection as shown in the heatmap of expression values in FIG. 4.

Using miRNAs to Predict BRCA Mutation Status

Having preselected 19 miRNAs with consistent capability of separating BRCA-mt from BRCA-wt samples, OmicSelector-based development of models were used to differentiate between BRCA-mut and wild-type samples based on batch-adjusted log 2(TPM) expression values. Feature sets derived from the training set Table 6 were used for modelling using four different approaches.

TABLE 6
Variable selection procedures used to identify a subset of
miRNAs with the best class separation properties.

| | | No. of |
|---|---|---|
| Name of Method | Included miRNAs | miRNAs |
| feseR_combineFS_RF | ~hsa-let-7b-5p, hsa-miR-320b, hsa-miR-30d-5p, | 7 |
| | hsa-miR-20b-5p, hsa-miR-19b-3p, hsa-miR-17- | |
| | 5p, hsa-miR-375-3p | |
| feseR_combineFS_RF_SMOTE | ~hsa-miR-320b, hsa-let-7b-5p, hsa-miR-19b-3p, | 8 |
| | hsa-miR-20b-5p, hsa-miR-30d-5p, hsa-miR-139- | |
| | 3p, hsa-miR-17-5p, hsa-miR-375-3p | |
| fcfsSMOTE | ~hsa-miR-20b-5p, hsa-miR-320b, hsa-let-7b-5p, | 10 |
| | hsa-miR-139-3p, hsa-miR-30d-5p, hsa-miR- | |
| | 106b-5p, hsa-miR-19b-3p, hsa-miR-1304-3p, | |
| | hsa-miR-421, hsa-miR-17-5p | |
| fcfs_sig | ~hsa-miR-20b-5p, hsa-miR-19b-3p, hsa-miR- | 7 |
| | 320b, hsa-let-7b-5p, hsa-miR-30d-5p, hsa-miR- | |
| | 139-3p, hsa-miR-106b-5p | |
| fcfsSMOTE_sig | ~hsa-miR-20b-5p, hsa-miR-320b, hsa-let-7b-5p, | 10 |
| | hsa-miR-139-3p, hsa-miR-30d-5p, hsa-miR- | |
| | 106b-5p, hsa-miR-19b-3p, hsa-miR-1304-3p, | |
| | hsa-miR-421, hsa-miR-17-5p | |
| fwrap | ~hsa-miR-1273h-3p, hsa-miR-182-5p, hsa-miR- | 4 |
| | 20b-5p, hsa-miR-320b | |
| fwrapSMOTE | ~hsa-miR-20b-5p, hsa-miR-421, hsa-miR-139- | 3 |
| | 3p | |
| fwrap_sig | ~hsa-miR-19b-3p, hsa-let-7b-5p, hsa-miR-182- | 5 |
| | 5p, hsa-miR-106b-5p, hsa-miR-134-5p | |
| fwrapSMOTE_sig | ~hsa-miR-20b-5p, hsa-miR-19b-3p, hsa-miR- | 7 |
| | 106b-5p, hsa-miR-4433a-3p, hsa-miR-500a-3p, | |
| | hsa-miR-485-3p, hsa-miR-1273h-3p | |
| AUC_MDL | ~hsa-miR-20b-5p, hsa-miR-19b-3p, hsa-let-7b- | 10 |
| | 5p, hsa-miR-320b, hsa-miR-30d-5p, hsa-miR- | |
| | 139-3p, hsa-miR-17-5p, hsa-miR-182-5p, hsa- | |
| | miR-421, hsa-miR-375-3p | |
| SU_MDL | ~hsa-miR-20b-5p, hsa-miR-19b-3p, hsa-let-7b- | 10 |
| | 5p, hsa-miR-139-3p, hsa-miR-320b, hsa-miR- | |
| | 30d-5p, hsa-miR-17-5p, hsa-miR-106b-5p, hsa- | |
| | miR-421, hsa-miR-182-5p | |
| CorrSF_MDL | ~hsa-miR-20b-5p, hsa-miR-19b-3p, hsa-miR- | 7 |
| | 320b, hsa-let-7b-5p, hsa-miR-30d-5p, hsa-miR- | |
| | 139-3p, hsa-miR-106b-5p | |
| sigtop | ~hsa-miR-20b-5p, hsa-miR-19b-3p, hsa-let-7b- | 10 |
| | 5p, hsa-miR-320b, hsa-miR-30d-5p, hsa-miR- | |
| | 17-5p, hsa-miR-139-3p, hsa-miR-182-5p, hsa- | |
| | miR-106b-5p, hsa-miR-375-3p | |
| sigtopBonf | ~hsa-miR-20b-5p, hsa-miR-19b-3p, hsa-let-7b- | 10 |
| | 5p, hsa-miR-320b, hsa-miR-30d-5p, hsa-miR- | |
| | 17-5p, hsa-miR-139-3p, hsa-miR-182-5p, hsa- | |
| | miR-106b-5p, hsa-miR-375-3p | |
| sigtopHolm | ~hsa-miR-20b-5p, hsa-miR-19b-3p, hsa-let-7b- | 10 |
| | 5p, hsa-miR-320b, hsa-miR-30d-5p, hsa-miR- | |
| | 17-5p, hsa-miR-139-3p, hsa-miR-182-5p, hsa- | |
| | miR-106b-5p, hsa-miR-375-3p | |
| topFC | ~hsa-miR-20b-5p, hsa-miR-375-3p, hsa-miR- | 10 |
| | 106b-5p, hsa-miR-19b-3p, hsa-miR-134-5p, hsa- | |
| | miR-17-5p, hsa-miR-320b, hsa-miR-139-3p, | |
| | hsa-miR-4433a-3p, hsa-miR-4433b-5p | |
| sigtopSMOTE | ~hsa-miR-20b-5p, hsa-miR-19b-3p, hsa-let-7b- | 10 |
| | 5p, hsa-miR-320b, hsa-miR-17-5p, hsa-miR- | |
| | 30d-5p, hsa-miR-139-3p, hsa-miR-182-5p, hsa- | |
| | miR-106b-5p, hsa-miR-375-3p | |
| sigtopBonfSMOTE | ~hsa-miR-20b-5p, hsa-miR-19b-3p, hsa-let-7b- | 10 |
| | 5p, hsa-miR-320b, hsa-miR-17-5p, hsa-miR- | |
| | 30d-5p, hsa-miR-139-3p, hsa-miR-182-5p, hsa- | |
| | miR-106b-5p, hsa-miR-375-3p | |
| sigtopHolmSMOTE | ~hsa-miR-20b-5p, hsa-miR-19b-3p, hsa-let-7b- | 10 |
| | 5p, hsa-miR-320b, hsa-miR-17-5p, hsa-miR- | |
| | 30d-5p, hsa-miR-139-3p, hsa-miR-182-5p, hsa- | |
| | miR-106b-5p, hsa-miR-375-3p | |
| topFCSMOTE | ~hsa-miR-20b-5p, hsa-miR-106b-5p, hsa-miR- | 10 |
| | 375-3p, hsa-miR-19b-3p, hsa-miR-134-5p, hsa- | |
| | miR-17-5p, hsa-miR-320b, hsa-miR-139-3p, | |
| | hsa-miR-493-5p, hsa-miR-182-5p | |
| AUC_MDLSMOTE | ~hsa-miR-20b-5p, hsa-miR-19b-3p, hsa-let-7b- | 10 |
| | 5p, hsa-miR-320b, hsa-miR-139-3p, hsa-miR- | |
| | 30d-5p, hsa-miR-17-5p, hsa-miR-182-5p, hsa- | |
| | miR-421, hsa-miR-375-3p | |
| SU_MDLSMOTE | ~hsa-miR-20b-5p, hsa-miR-19b-3p, hsa-let-7b- | 10 |
| | 5p, hsa-miR-320b, hsa-miR-139-3p, hsa-miR- | |
| | 106b-5p, hsa-miR-17-5p, hsa-miR-30d-5p, hsa- | |
| | miR-421, hsa-miR-182-5p | |
| CorrSF_MDLSMOTE | ~hsa-miR-20b-5p, hsa-miR-320b, hsa-let-7b-5p, | 10 |
| | hsa-miR-139-3p, hsa-miR-30d-5p, hsa-miR- | |
| | 106b-5p, hsa-miR-19b-3p, hsa-miR-1304-3p, | |
| | hsa-miR-421, hsa-miR-17-5p | |
| AUC_MDL_sig | ~hsa-miR-20b-5p, hsa-miR-19b-3p, hsa-let-7b- | 10 |
| | 5p, hsa-miR-320b, hsa-miR-30d-5p, hsa-miR- | |
| | 139-3p, hsa-miR-17-5p, hsa-miR-182-5p, hsa- | |
| | miR-421, hsa-miR-375-3p | |
| SU_MDL_sig | ~hsa-miR-20b-5p, hsa-miR-19b-3p, hsa-let-7b- | 10 |
| | 5p, hsa-miR-139-3p, hsa-miR-320b, hsa-miR- | |
| | 30d-5p, hsa-miR-17-5p, hsa-miR-106b-5p, hsa- | |
| | miR-421, hsa-miR-182-5p | |
| CorrSF_MDL_sig | ~hsa-miR-20b-5p, hsa-miR-19b-3p, hsa-miR- | 7 |
| | 320b, hsa-let-7b-5p, hsa-miR-30d-5p, hsa-miR- | |
| | 139-3p, hsa-miR-106b-5p | |
| AUC_MDLSMOTE_sig | ~hsa-miR-20b-5p, hsa-miR-19b-3p, hsa-let-7b- | 10 |
| | 5p, hsa-miR-320b, hsa-miR-139-3p, hsa-miR- | |
| | 30d-5p, hsa-miR-17-5p, hsa-miR-182-5p, hsa- | |
| | miR-421, hsa-miR-375-3p | |
| SU_MDLSMOTE_sig | ~hsa-miR-20b-5p, hsa-miR-19b-3p, hsa-let-7b- | 10 |
| | 5p, hsa-miR-320b, hsa-miR-139-3p, hsa-miR- | |
| | 106b-5p, hsa-miR-17-5p, hsa-miR-30d-5p, hsa- | |
| | miR-421, hsa-miR-182-5p | |
| CorrSF_MDLSMOTE_sig | ~hsa-miR-20b-5p, hsa-miR-320b, hsa-let-7b-5p, | 10 |
| | hsa-miR-139-3p, hsa-miR-30d-5p, hsa-miR- | |
| | 106b-5p, hsa-miR-19b-3p, hsa-miR-1304-3p, | |
| | hsa-miR-421, hsa-miR-17-5p | |
| fcsig | ~hsa-miR-20b-5p, hsa-miR-106b-5p, hsa-miR- | 3 |
| | 375-3p | |
| fcsigSMOTE | ~hsa-miR-20b-5p, hsa-miR-106b-5p | 2 |
| RandomForestRFE | ~hsa-miR-320b, hsa-let-7b-5p, hsa-miR-20b-5p, | 10 |
| | hsa-miR-30d-5p, hsa-miR-19b-3p, hsa-miR-139- | |
| | 3p, hsa-miR-375-3p, hsa-miR-17-5p, hsa-miR- | |
| | 1304-3p, hsa-miR-106b-5p | |
| RandomForestRFE_sig | ~hsa-miR-320b, hsa-let-7b-5p, hsa-miR-20b-5p, | 10 |
| | hsa-miR-30d-5p, hsa-miR-19b-3p, hsa-miR-139- | |
| | 3p, hsa-miR-375-3p, hsa-miR-17-5p, hsa-miR- | |
| | 106b-5p, hsa-miR-1304-3p | |
| RandomForestRFESMOTE_sig | ~hsa-miR-320b, hsa-let-7b-5p, hsa-miR-30d-5p, | 9 |
| | hsa-miR-20b-5p, hsa-miR-19b-3p, hsa-miR-139- | |
| | 3p, hsa-miR-375-3p, hsa-miR-1304-3p, hsa- | |
| | miR-106b-5p | |
| cfs | ~hsa-miR-19b-3p, hsa-let-7b-5p, hsa-miR-106b- | 7 |
| | 5p, hsa-miR-30d-5p, hsa-miR-20b-5p, hsa-miR- | |
| | 320b, hsa-miR-139-3p | |
| cfsSMOTE | ~hsa-miR-19b-3p, hsa-let-7b-5p, hsa-miR-106b- | 10 |
| | 5p, hsa-miR-17-5p, hsa-miR-1304-3p, hsa-miR- | |
| | 30d-5p, hsa-miR-20b-5p, hsa-miR-421, hsa- | |
| | miR-320b, hsa-miR-139-3p | |
| cfs_sig | ~hsa-miR-20b-5p, hsa-miR-19b-3p, hsa-let-7b- | 7 |
| | 5p, hsa-miR-320b, hsa-miR-30d-5p, hsa-miR- | |
| | 139-3p, hsa-miR-106b-5p | |
| cfsSMOTE_sig | ~hsa-miR-20b-5p, hsa-miR-19b-3p, hsa-let-7b- | 10 |
| | 5p, hsa-miR-320b, hsa-miR-30d-5p, hsa-miR- | |
| | 17-5p, hsa-miR-139-3p, hsa-miR-106b-5p, hsa- | |
| | miR-421, hsa-miR-1304-3p | |
| SimulatedAnnealingRF | ~hsa-miR-19b-3p, hsa-miR-134-5p, hsa-miR- | 6 |
| | 182-5p, hsa-miR-20b-5p, hsa-miR-421, hsa- | |
| | miR-375-3p | |
| SimulatedAnnealingRFSMOTE | ~hsa-miR-485-3p, hsa-let-7b-5p, hsa-miR-134- | 6 |
| | 5p, hsa-miR-1304-3p, hsa-miR-4433b-5p, hsa- | |
| | miR-139-3p | |
| Mystepwise_glm_binomial | ~hsa-miR-19b-3p, hsa-miR-4433a-3p, hsa-let- | 8 |
| | 7b-5p, hsa-miR-106b-5p, hsa-miR-1304-3p, hsa- | |
| | miR-30d-5p, hsa-miR-320b, hsa-miR-375-3p | |
| Mystepwise_sig_glm_binomial | ~hsa-miR-19b-3p, hsa-miR-4433a-3p, hsa-let- | 8 |
| | 7b-5p, hsa-miR-106b-5p, hsa-miR-1304-3p, hsa- | |
| | miR-30d-5p, hsa-miR-320b, hsa-miR-375-3p | |
| ElasticNet | ~hsa-let-7b-5p, hsa-miR-30d-5p, hsa-miR-320b, | 8 |
| | hsa-miR-19b-3p, hsa-miR-20b-5p, hsa-miR- | |
| | 1304-3p, hsa-miR-139-3p, hsa-miR-375-3p | |
| classloop_sig | ~hsa-miR-20b-5p, hsa-miR-19b-3p, hsa-let-7b- | 10 |
| | 5p, hsa-miR-320b, hsa-miR-30d-5p, hsa-miR- | |
| | 17-5p, hsa-miR-139-3p, hsa-miR-182-5p, hsa- | |
| | miR-375-3p, hsa-miR-421 | |
| | ~hsa-miR-20b-5p, hsa-miR-19b-3p, hsa-miR- | |
| | 320b, hsa-let-7b-5p, hsa-miR-30d-5p, hsa-miR- | |
| fcfs | 139-3p, hsa-miR-106b-5p | 7 |

The best predictive performance was achieved by a logistic regression model with parameters shown in Table 7 based on 10 miRNAs: hsa-miR-20b-5p, hsa-miR-19b-3p, hsa-let-7b-5p, hsa-miR-320b, hsa-miR-139-3p, hsa-miR-30d-5p, hsa-miR-17-5p, hsa-miR-182-5p, hsa-miR-421, and hsa-miR-375-3p.

TABLE 7
Confusion matrix of the final classification model used in Example 1.

| | | BRCAwt | BRCAmut | Sensitivity | Specificity | PPV | NPV |
|---|---|---|---|---|---|---|---|
| Training | Predicted | 131 | 35 | 84.51% | 79.39% | 84.89% | 78.92% |
| set | BRCAwt | | | | | | |
| | Predicted | 34 | 191 | | | | |
| | BRCAMut | | | | | | |
| Testing | Predicted | 46 | 11 | 85.33% | 83.64% | 87.67% | 80.70% |
| set | BRCAwt | | | | | | |
| | Predicted | 9 | 64 | | | | |
| | BRCAMut | | | | | | |
| Validation | Predicted | 67 | 3 | 93.88% | 80.72% | 74.19% | 95.71% |
| set | BRCAwt | | | | | | |
| | Predicted | 16 | 46 | | | | |
| | BRCAMut | | | | | | |

The dataset is split into three parts to establish what variable does what on the training set and provide a first list of parameters. Therefore, the validation set provides a modicum of “independent validation” as those samples are not used in the training process at all. This set of miRNAs was selected using feature ranking based on ROC AUC and a minimal description length (MDL) discretization algorithm on the training set balanced with the Synthetic Minority Oversampling Technique (SMOTE).

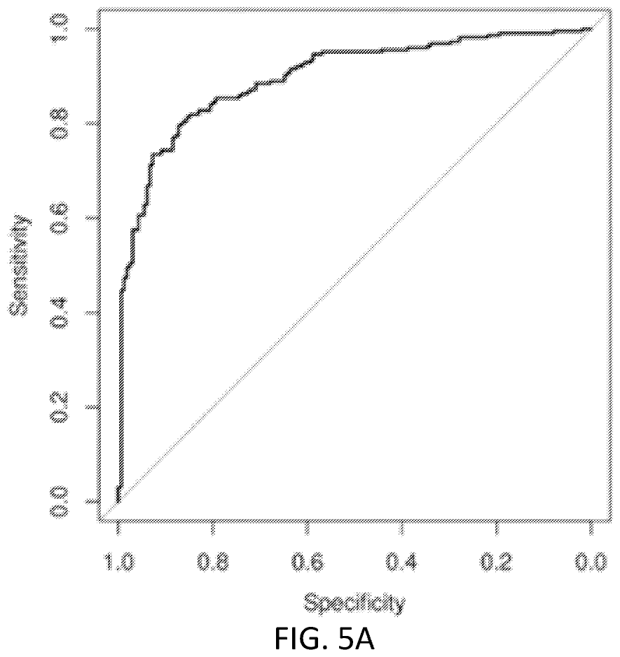

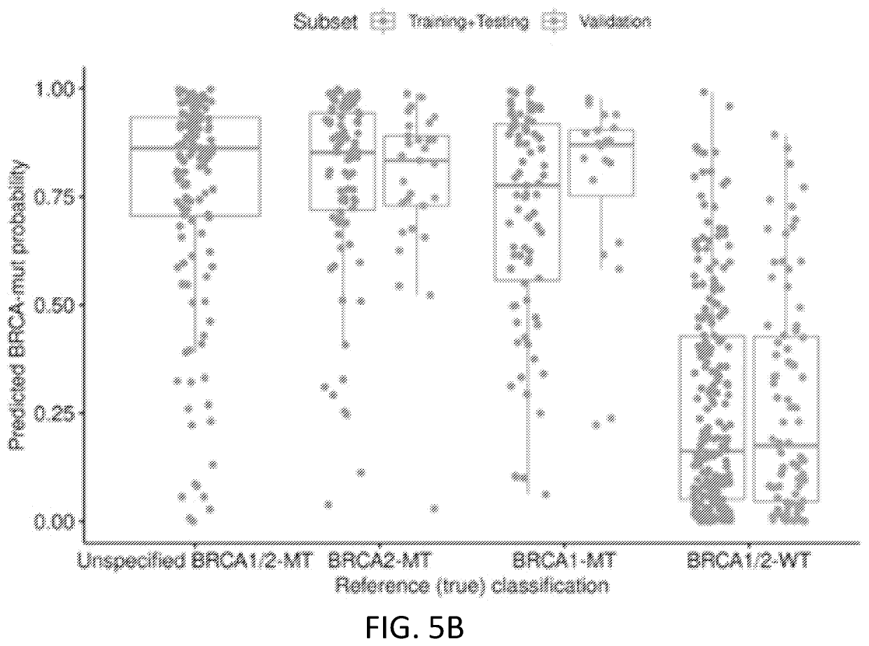

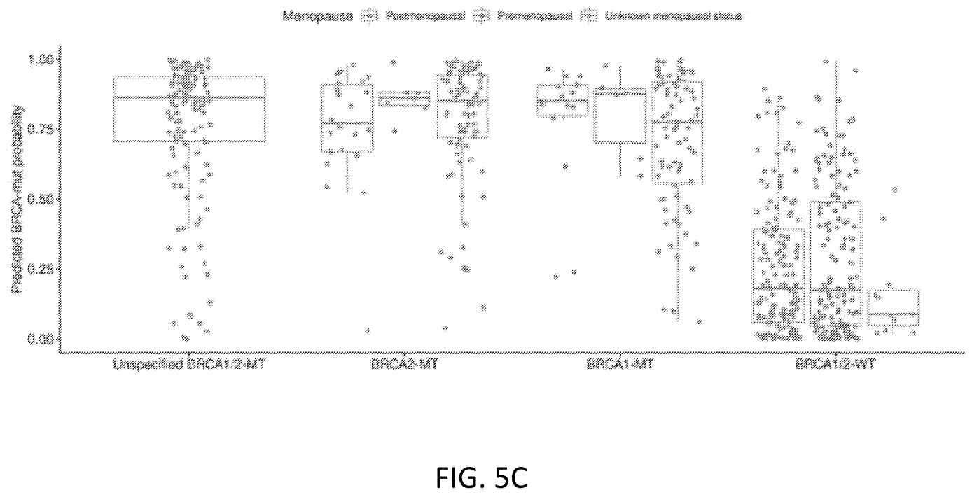

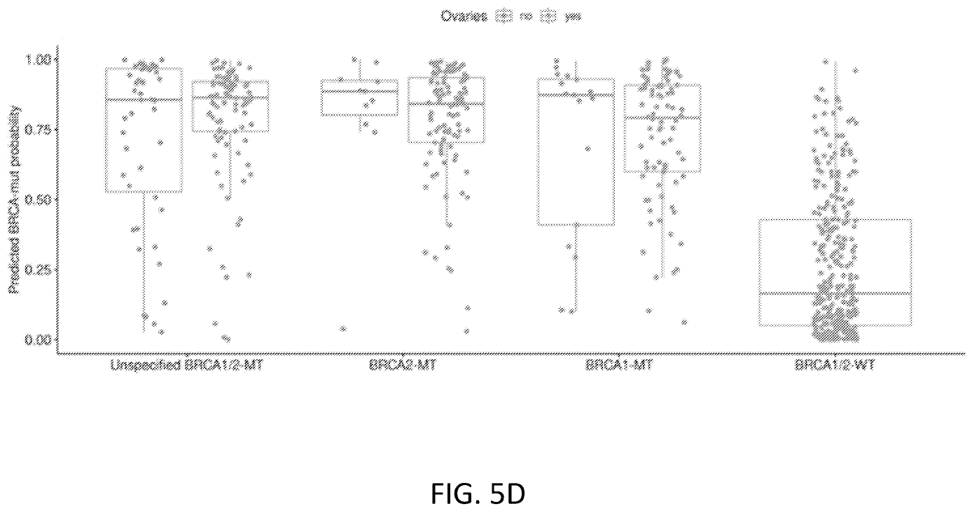

The final model achieved 82.35% accuracy, 84.51% sensitivity and 79.39% specificity on the original training set. The training AUC ROC (FIG. 5A) was 0.89 (95% CI: 0.87-0.93). This model achieved 84.62% accuracy, 95.33% sensitivity and 83.64% specificity on the testing set and 85.61% accuracy, 93.88% sensitivity and 80.72% specificity in the external validation set included of the UPenn group. Confusion matrices for the separate sets were obtained. Predicted probabilities of BRCA-mt in the context of true BRCA status are presented in FIG. 5B. Menopausal status (FIG. 5C) or having preemptive oophorectomy before blood sample draw (FIG. 5D) did not affect classification performance. The presented diagnostic performance was calculated for the cut-off established on the basis of optimal accuracy. However, to better evaluate the utility of the proposed test, the estimated positive and negative predictive values of different thresholds (based on the results from the whole patient cohort) for populations of varying prevalence of mutations in genes associated with homologous recombination pathway of DNA repair were present. Although data on accurate age at testing was provided for 52% of subjects, with a predominance of controls. The model's performance remained constant throughout the whole range of age categories.

### Example 2

A method for employing a joint lasso dimensionality reduction (DR) approach combined with a linear classification model to classify patients as likely BRCA mutation carriers or non-carriers is described below. The model incorporates both serum miRNA and clinical metadata and provides a score of “BRCAness.” BRCAness can serve as a surrogate biomarker for near-term ovarian cancer risk. The model described below is novel for incorporating clinical metadata and a miRNA assessment of BRCAness and is unique in its application for forecasting ovarian cancer risk. This can be used as a clinical test to estimate an individual's future risk of ovarian cancer and as a screening test prior to referral for genetic testing.

Hereditary breast and ovarian cancer syndrome (HBOC) are characterized by markedly increased lifetime risks of breast and ovarian cancer as well as melanoma, prostate, and pancreatic cancers. The most commonly mutated genes in HBOC are the DNA homologous repair (HR) genes BRCA1 and BRCA2, although mutations in a number of other moderate penetrance genes involved in HR can present a similar phenotype. Currently, the only clinical indications for genetic testing for HBOC are a personal history of a HBOC-related cancer, a strong family history of cancer which might suggest HBOC, or a close family relation with a known genetic mutation linked to HBOC. Routine testing for HBOC is not recommended. As a result, only 10% of the estimated 1 million BRCA1 or BRCA2 mutation carriers in the United States are aware of their mutation status and likely even lower percentages among the moderate penetrance genes. Compounding this lack of awareness, conventional BRCA1 and BRCA2 testing panels have historically had poor representation of genetic variants seen in non-white European populations, leading to higher rates of variants of unknown significance (VUS) among non-white European racial and ethnic groups.

Recent data indicates that germline BRCA1/2 mutation carriers without cancer have distinct circulating microRNA (miRNA) profiles from non-carriers. In that report, a model incorporating 10 miRNAs selected from next generation sequencing (NGS) data achieved 93.9% sensitivity at 80.7% specificity for identifying mutation carriers in an independent validation set. However, NGS remains a resource-constrained technology for widespread implementation into clinical practice. Moreover, risk assessment does not occur in a vacuum from individual subject factors. In the following example, additional miRNA panels were evaluated as screens for BRCA1/2 mutations. This functional assessment of BRCAness is tested to determine whether it is clinically informative for estimating the 5-year risk of ovarian cancer using an independent, prospective cohort.

### Results

### Patient Population

Demographic characteristics of the study population are shown in Table 8. Study subjects included n=1,831 women, the majority of which (95%) lived within a zip code in Massachusetts, receiving routine primary or specialty medical care within a facility affiliated with Mass General Brigham, a large New England healthcare system. Among study subjects, n=100 were known germline BRCA1/2 mutation carriers and n=1,731 were either confirmed non-carriers (n=159) or are had not undergone germline testing (n=1,572). BRCA1/2 mutation carriers were more likely to be white or post-menopausal. Not surprisingly, mutation carriers were more likely to have a personal or family history of breast or ovarian cancer and had higher prevalence of benign breast disease. Mutation carriers and non-carriers were similar in terms of parity, number of abortions, ectopic pregnancies, smoking history, BMI, and gynecologic history. All variables listed in Table 8 are used to train the classification model, with the exception of oral contraceptive use and hysterectomy. These variables are omitted from the model training due to potential reverse causality. For example, BRCA1 mutation carriers in this study may have undergone hysterectomy as part of risk-reducing surgery to minimize cancer risk.

TABLE 8
Characteristics of the training population. The p-values in the right-hand column
indicate if there is a statistically significant difference based on a two-sample t-test.
In each case, the number of subjects belonging to each class and the proportion are
given in parentheses. For some variables (e.g., parity), the data was missing for
some subjects. In the first column in parenthesis, the number of samples (n)
where data was recorded for each variable is provided.

| Variables | non-BRCA | BRCA | p-value |
|---|---|---|---|
| Smoking history (n = 1829) | 604 (34.9%) | 32 (32%) | 0.5493 |
| Obesity (BMI > 30) (n = 1831) | 559 (32.3%) | 30 (30%) | 0.6331 |
| Height (>average) (n = 1829) | 705 (40.8%) | 38 (38%) | 0.5828 |
| Parity (>0) (n = 1770) | 1108 (66.1%) | 65 (68.4%) | 0.6487 |
| Abortions (>0) (n = 1426) | 309 (23%) | 20 (24.7%) | 0.7216 |
| Ectopic pregnancies (>0) (n = 1700) | 39 (2.4%) | 4 (4.3%) | 0.2729 |
| Hormone replacement therapy use | 219 (13.7%) | 14 (15.7%) | 0.5848 |
| (choice = ever) (n = 1690) | | | |
| Oral contraceptive use (choice = ever) (n = 1831) | 648 (37.4%) | 55 (55%) | 0.0004 |
| Tubal ligation (n = 1819) | 241 (14%) | 10 (10%) | 0.2572 |
| Benign gynecologic disease (n = 1831) | 347 (20%) | 22 (22%) | 0.6358 |
| Endometriosis (n = 1802) | 196 (11.5%) | 15 (15%) | 0.2923 |
| Hysterectomy (n = 1826) | 254 (14.7%) | 36 (36.4%) | <0.0001 |
| Benign breast disease (n = 1816) | 370 (21.5%) | 37 (37.4%) | 0.0002 |
| Ovarian cancer in family (first degree) (n = 1831) | 32 (1.8%) | 11 (11%) | <0.0001 |
| Breast cancer in family (first degree) (n = 1831) | 159 (9.2%) | 36 (36%) | <0.0001 |
| Coronary artery disease (choice = yes) (n = 1831) | 80 (4.6%) | 4 (4%) | 0.7727 |
| Gallbladder disease (choice = yes) (n = 1831) | 277 (16%) | 16 (16%) | 0.9995 |
| Colon polyps (choice = yes) (n = 1831) | 343 (19.8%) | 22 (22%) | 0.5949 |
| Hypertension (choice = yes) (n = 1831) | 576 (33.3%) | 29 (29%) | 0.3768 |
| Osteoporosis (choice = yes) (n = 1831) | 193 (11.1%) | 19 (19%) | 0.0171 |
| Diabetes mellitus (choice = yes) (n = 1831) | 177 (10.2%) | 9 (9%) | 0.6933 |

### Classification Results

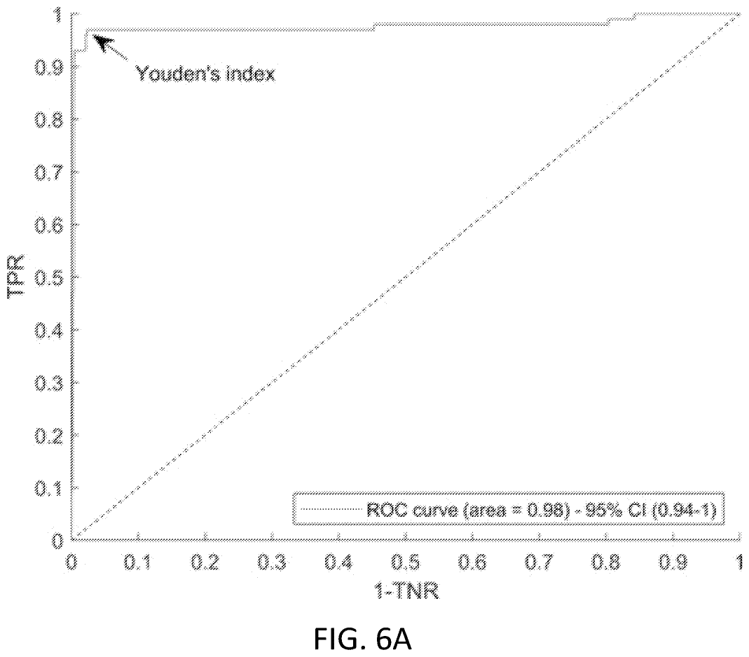

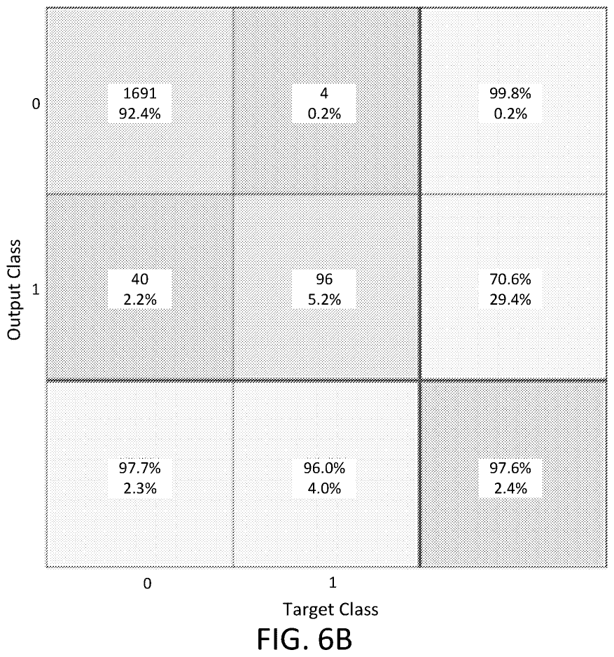

The clinical metadata were combined with miRNA expression data from a focused panel of 179 serum miRNAs using a joint lasso dimension reduction technique to classify study subjects as likely BRCA1/2 mutation carriers or non-carriers (FIG. 6A). See the methods section below for a full description of the joint lasso model. See Table 9 for a full list of all 179 miRNAs considered in this study. After 10-fold cross validation, the area under the curve (AUC) of the receiver operator characteristic (ROC) curve was 0.98 (95% CI 0.94-1) (note: the predictions for all 10 folds were concatenated together and evaluated against the full set of true BRCA labels). The number of non-zero coefficients in the miRNA component of the model was 151 (84% of miRNAs), and the metadata component had 11 non-zero coefficients (58% of metadata variables considered).

TABLE 9
Full list of all 179 miRNA considered in this example.

| | | |
|---|---|---|
| hsa-mir-185-5p | hsa-let-7d-5p | hsa-mir-99a-5p |
| hsa-mir-320e | hsa-mir-17-5p | hsa-mir-532-3p |
| hsa-mir-181b-5p | hsa-mir-425-5p | hsa-mir-409-3p |
| hsa-mir-194-5p | hsa-mir-93-5p | hsa-mir-99b-5p |
| hsa-mir-486-5p | hsa-mir-16-5p | hsa-mir-495-3p |
| hsa-mir-145-5p | hsa-mir-191-5p | hsa-mir-148b-3p |
| hsa-mir-422a | hsa-mir-1237-3p | hsa-mir-126-5p |
| hsa-mir-148a-3p | hss-mir-26a-5p | hsa-mir-590-5p |
| hsa-let-7f-5p | hsa-mir-589-6p | hsa-mir-199b-3p |
| hsa-mir-20b-5p | hsa-mir-345-5p | hsa-mir-181a-5p |
| hsa-mir-199a-3p | hsa-mir-375-3p | hsa-mir-30a-5p |
| hsa-mir-151a-5p | hsa-let-7e-5p | hsa-mir-574-5p |
| hsa-mir-34c-5p | hsa-mir-28-5p | hsa-mir-378a-3p |
| hsa-mir-154-5p | hsa-mir-100-5p | hsa-mir-497-5p |
| hsa-mir-766-3p | hsa-mir-206 | hsa-mir-19a-3p |
| hsa-mir-324-3p | hsa-mir-423-3p | hsa-mir-134-5p |
| hsa-mir-133b | hsa-mir-181d-5p | hsa-mir-382-5p |
| hsa-mir-1260a | hsa-mir-140-3p | hsa-mir-34a-5p |
| hsa-mix-195-5p | hsa-mir-21-5p | hsa-mir-424-5p |
| hsa-mir-503-5p | hsa-mir-24-3p | hsa-mir-376c-3p |
| hsa-mir-103a-3p | hsa-mir-210-3p | hsa-mir-222-3p |
| hsa-mir-92a-3p | hsa-mir-885-5p | hsa-mir-483-5p |
| hsa-mir-223-3p | hsa-mir-133a-3p | hsa-mir-32-5p |
| hsa-mir-205-5p | hsa-mir-193a-5p | hsa-let-7b-5p |
| hsa-mir-30e-5p | hsa-mir-320b | hsa-mir-431-3p |
| hsa-mir-139-3p | hsa-mir-200c-3p | hsa-mir-320d |
| hsa-mir-378c | hsa-mir-17-3p | hsa-mir-98-5p |
| hsa-mir-144-3p | hsa-mir-130a-3p | hsa-mir-15b-5p |
| hsa-mir-29a-3p | hsa-mir-363-3p | hsa-mir-185-3p |
| hsa-mir-25-3p | hsa-mir-34b-3p | hsa-mir-29b-3p |
| hsa-mir-584-5p | hsa-mir-33a-5p | hsa-mir-186-5p |
| hsa-mir-18a-5p | hsa-mir-28-3p | hsa-mir-20a-5p |
| hsa-mir-106b-3p | hsa-mir-142-5p | hsa-let-7g-5p |
| hsa-mir-487b-3p | hsa-mir-744-5p | hsa-mir-187-3p |
| hsa-mir-15a-5p | hsa-mir-323a-3p | hsa-mir-128-3p |
| hsa-mir-10b-5p | hsa-mir-30d-5p | hsa-mir-629-5p |
| hsa-let-7d-3p | hsa-mir-373-5p | hsa-mir-502-3p |
| hsa-mir-132-3p | hsa-mir-4433b-3p | hsa-mir-326 |
| hsa-mir-328-3p | hsa-mir-708-5p | hsa-mir-130b-3p |
| hsa-mir-221-3p | hsa-mir-26b-5p | hsa-mir-150-5p |
| hsa-mir-323b-3p | hsa-mir-324-5p | hsa-mir-532-5p |
| hsa-mir-152-3p | hsa-mir-183-5p | hsa-mir-30c-5p |
| hsa-mir-103a-2-5p | hsa-mir-126-3p | hsa-mir-877-5p |
| hsa-mir-484 | hsa-mir-770-5p | hsa-mir-501-3p |
| hsa-mir-106b-5p | hsa-mir-361-5p | hsa-mir-151b |
| hsa-mir-320c | hsa-mir-92b-3p | hsa-mir-4732-5p |
| hsa-mir-404-3p | hsa-let-7i-5p | hsa-mir-140-5p |
| hsa-mir-23a-3p | hsa-mir-454-3p | hsa-mir-27a-3p |
| hsa-mir-486-3p | hsa-mir-301a-3p | hsa-mir-146b-5p |
| hsa-mir-335-5p_0_-2 | hsa-mir-421 | hsa-mir-143-3p |
| hsa-mir-107 | hsa-mir-342-3p | hsa-mir-29c-3p |
| hsa-mir-27b-3p | hsa-mir-196b-5p | hsa-mir-342-5p |
| hsa-mir-451a | hsa-mir-146a-5p | hsa-mir-660-5p |
| hsa-let-7c-5p | hsa-mir-181c-5p | hsa-mir-7-1-3p |
| hsa-mir-423-5p | hsa-mir-125b-5p | hsa-mir-18b-5p |
| hsa-mir-671-3p | hsa-mir-652-3p | hsa-mir-211-5p |
| hsa-mir-106a-5p | hsa-mir-877-3p | hsa-mir-199a-5p |
| hsa-mir-1260b | hsa-mir-338-3p | hsa-mir-22-5p |
| hsa-mir-574-3p | hsa-mir-320a-3p | hsa-mir-23b-3p |
| hsa-let-7a-5p | hsa-mir-122-5p | |

Next, the effects of further limiting the number of input variables were investigated. The subject was classified as a BRCA mutation carrier if their BRCA mutation probability was greater than t∈[0,1], and conversely as a non-BRCA mutation carrier if the probability of BRCA mutation was less than or equal to t. FIG. 6B shows the confusion matrix corresponding to t=0.04, which is the t that corresponds to Youden's index. Different shading is used to show cases in which the target class and output class are the same (e.g., 0,0 or 1,1) and different (e.g., 0.1 or 1,0). The specificity of the model is 98%, the sensitivity is 96%, and the overall classification accuracy is ACC=98%. For example, for a first-pass test for BRCA mutation, where the goal was to identify ≥95% of BRCA carriers, then one could set t=0.04 as in FIG. 6B. In this case, 98% of the control group, which equates to 93% of the overall population, can be safely excluded from further testing, as the probability of BRCA mutation for these subjects is low (i.e., <0.04). The remaining 7% of subjects, which are more likely BRCA1/2 carriers, can be moved onto confirmatory genetic testing. The positive predictive value in this population is 71%. Similarly, t can be varied to favor specificity or sensitivity, depending on the application.

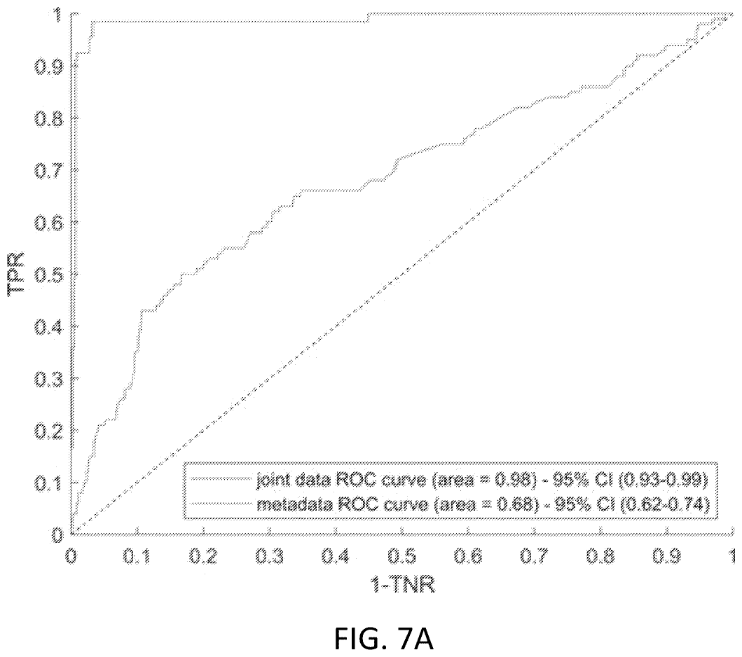

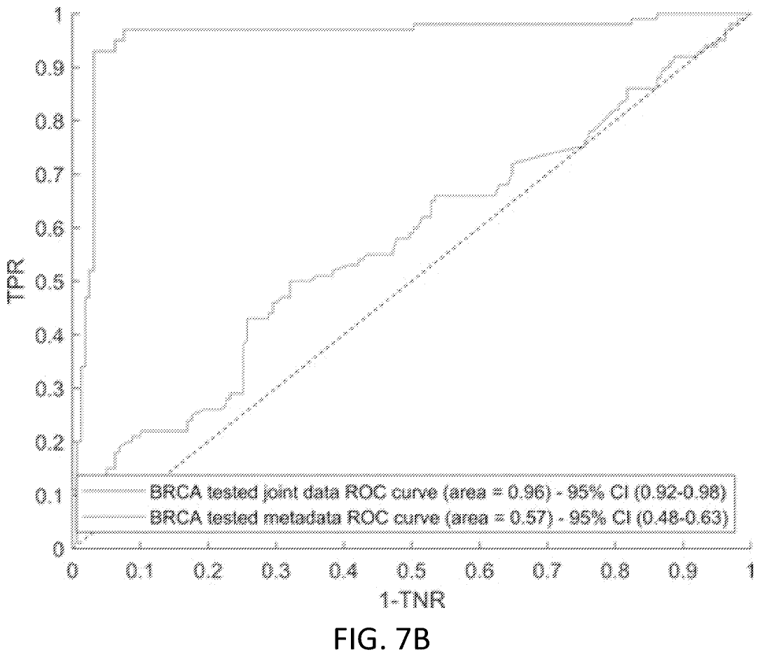

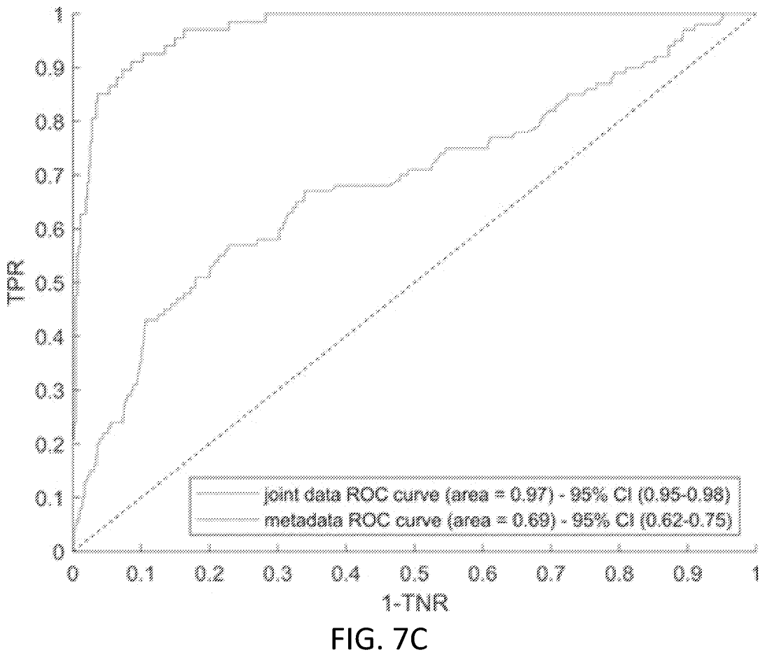

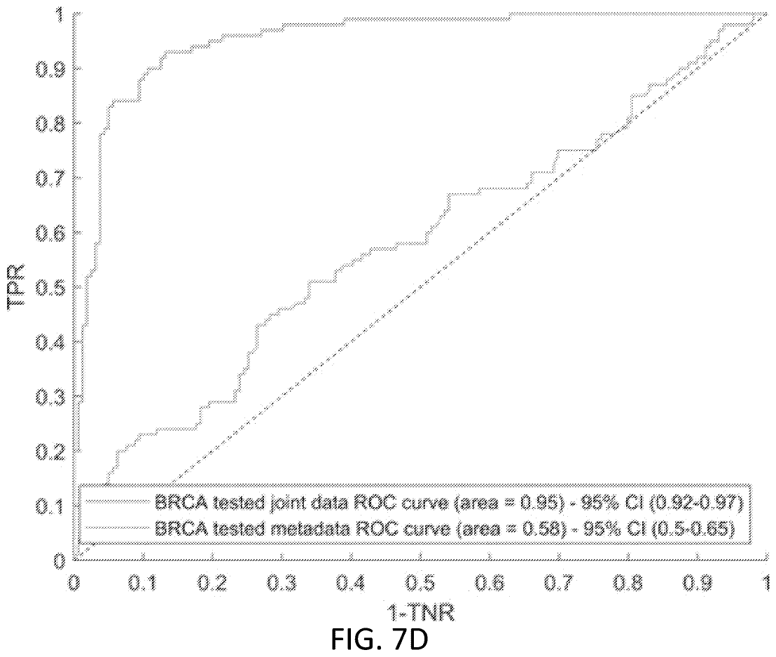

In FIG. 7A, the joint lasso model (which fuses miRNA and metadata) is compared to the metadata component alone, as baseline, to show the benefits of the combined model. After 10-fold cross-validation, the metadata model offers an AUC=0.68 (95% CI 0.62-0.74), which is 30% lower than the AUC=0.98 (95% CI 0.94-1) offered by the joint model, and thus including miRNA in the joint lasso model is shown to significantly improve classification performance.

### Effects of Varying the Number of Model Input Variables

The joint lasso model retained 151 out of 179 miRNAs when the lasso parameter was selected using cross-validation.

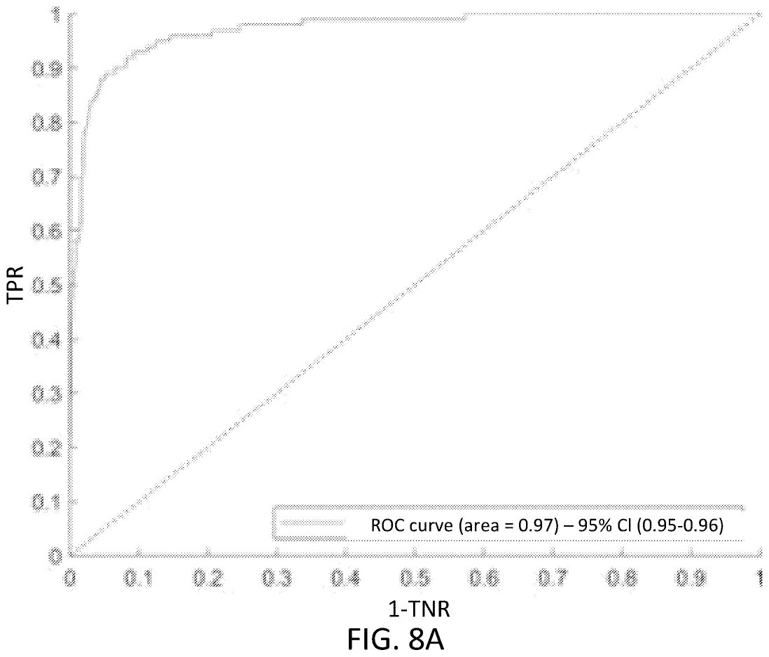

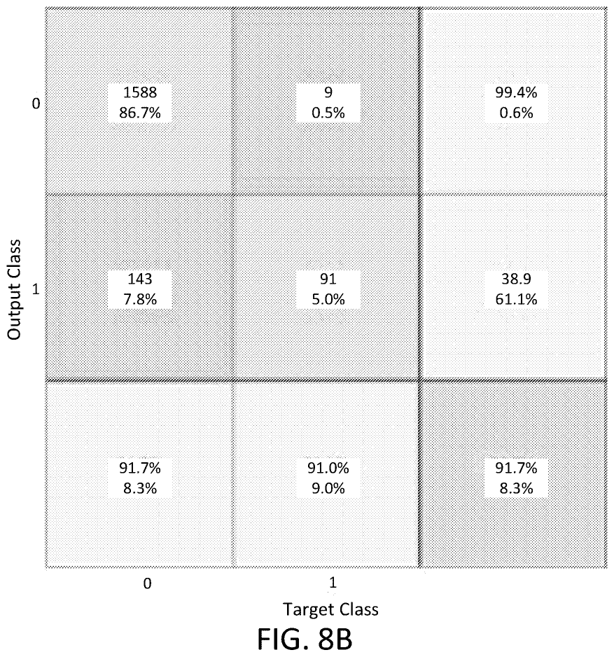

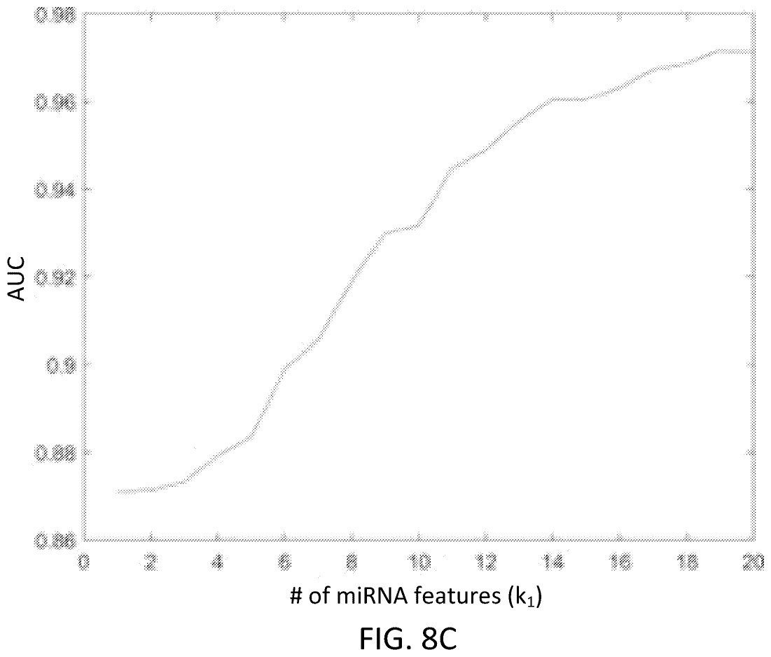

Model performance was examined based on varying the number of miRNA inputs (k₁), and metadata features (k₂). A subset of k₁≤20 miRNA and k₂≤20 metadata features were selected with strong linear relation to BRCA status (as listed in Table 8). To do this, the number of non-zero lasso components, e.g., the non-zero entries of v₁ and v₂ (see the methods section), was restricted to a maximum of k₁ and k₂, respectively. The model performance was measured after 10-fold cross-validation for k₁∈{1, . . . , 20} and k₂∈{1, . . . , 19} (k₂=19 is the upper limit as this is the total number of metadata features used to train the joint lasso model). In terms of AUC score, there was not an improvement in performance as k₂ increased past k₂=5, and the AUC increases monotonically with k₁. FIG. 8C, shows how the model performance varies in terms of AUC with k₁, in the case when k₂=5. The AUC score is maximized at AUC=0.97. For example, when k₁=20 and k₂=5, the AUC score is AUC=0.97 (95% CI 0.95-0.98). FIG. 8A shows a plot of the ROC curve corresponding to the k₁=20 miRNA and k₂=5 metadata feature model. The k₁=20 miRNA and k₂=5 metadata features chosen by the lasso model are listed in Tables 10 and 11, respectfully. FIG. 8B shows the confusion matrix corresponding to the Youden index, as in FIG. 6A. Thus, by limiting k₁=20 and k₂=5, little performance is sacrificed in terms of AUC. In this case, the specificity offered by the model is 92%, and the sensitivity is 91%, with overall accuracy ACC=92%, which indicates excellent performance. The positive predictive value of the model is also 38.9% (95% CI 33.4%-43.6%) with a negative predictive value of 99.4% (95% CI 99.0-99.7%).

TABLE 11
List of k₂ = 5 metadata features chosen by lasso. Statistics
for these variables are included in Table 8.
Height
Benign breast disease
Ovarian cancer in family (first degree)
Breast cancer in family (first degree)
Osteoporosis (choice = yes)

TABLE 10
The selected k₁ = 20 miRNA features using lasso. The mean (μ) and
standard deviation (σ) log2 expression are shown for the non-BRCA
and BRCA subjects, along with fold change values, and p values
indicating if the difference in means is significant. The fold change
is defined as the mean non-BRCA expression divided by the mean
BRCA expression, e.g., 0.96 = 5.95/6.2 on row 1.

| miRNA | μ ± σ (non-BRCA) | μ ± σ (BRCA) | fold change | p value |
|---|---|---|---|---|
| hsa-mir-34c-5p | 5.95 ± 0.97 | 6.2 ± 0.93 | 0.96 | 0.009 |
| hsa-mir-133b | 6.56 ± 1.09 | 6.74 ± 0.9 | 0.97 | 0.05 |
| hsa-mir-223-3p | 15.4 ± 1.03 | 14.43 ± 1.32 | 1.07 | <0.001 |
| hsa-mir-18a-5p | 11.04 ± 1.21 | 9.86 ± 1.14 | 1.12 | <0.001 |
| hsa-mir-484 | 13.18 ± 1.06 | 13.13 ± 1.17 | 1.004 | 0.712 |
| hsa-mir-425-5p | 13.26 ± 0.94 | 12.01 ± 1.19 | 1.1 | <0.001 |
| hsa-mir-1237-3p | 8.21 ± 1.31 | 7.1 ± 0.59 | 1.16 | <0.001 |
| hsa-mir-100-5p | 9.37 ± 1.3 | 9.58 ± 1.25 | 0.98 | 0.103 |
| hsa-mir-133a-3p | 7.33 ± 1.27 | 6.02 ± 1.07 | 1.22 | <0.001 |
| hsa-mir-130a-3p | 14.1 ± 1.28 | 14.39 ± 1.24 | 0.98 | 0.029 |
| hsa-mir-770-5p | 7.94 ± 0.91 | 8.24 ± 0.9 | 0.96 | 0.002 |
| hsa-mir-125b-5p | 5.55 ± 0.77 | 5.82 ± 0.86 | 0.95 | 0.003 |
| hsa-mir-338-3p | 8.38 ± 1.8 | 6.51 ± 1.85 | 1.29 | <0.001 |
| hsa-mir-34a-5p | 9.28 ± 1.25 | 7.87 ± 1.39 | 1.18 | <0.001 |
| hsa-mir-424-5p | 10.42 ± 1.87 | 8.54 ± 2.06 | 1.22 | <0.001 |
| hsa-mir-185-3p | 9.17 ± 1.19 | 7.3 ± 1.47 | 1.26 | <0.001 |
| hsa-mir-629-5p | 10.4 ± 1.39 | 10.13 ± 1.39 | 1.03 | 0.058 |
| hsa-mir-130b-3p | 12.71 ± 1.21 | 12.32 ± 1.51 | 1.03 | 0.012 |
| hsa-mir-660-5p | 11.8 ± 1.63 | 11.63 ± 1.64 | 1.02 | 0.106 |

### Performance of a BRCA1/2 Classifier Across Race, Age, or Cancer Status Subgroups

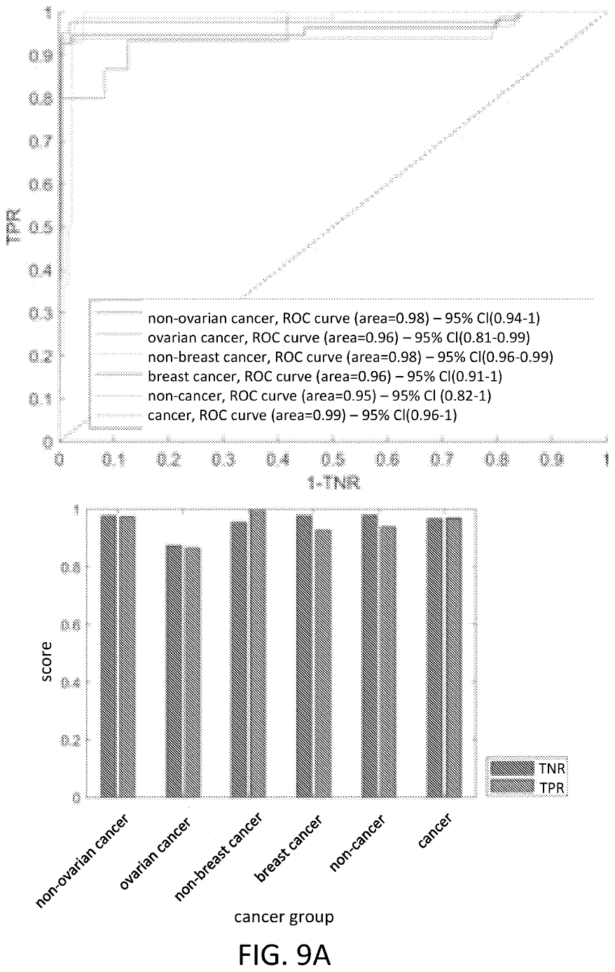

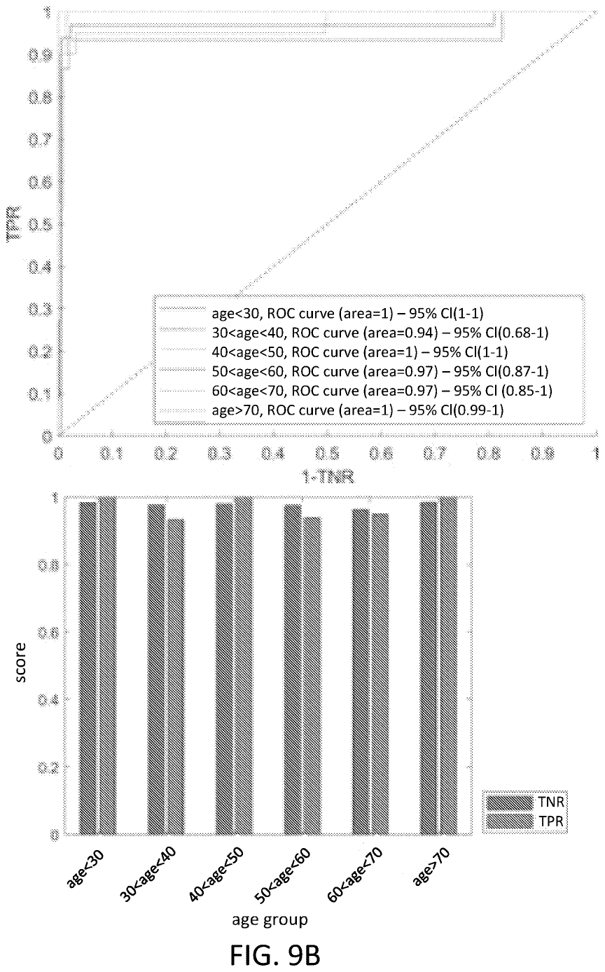

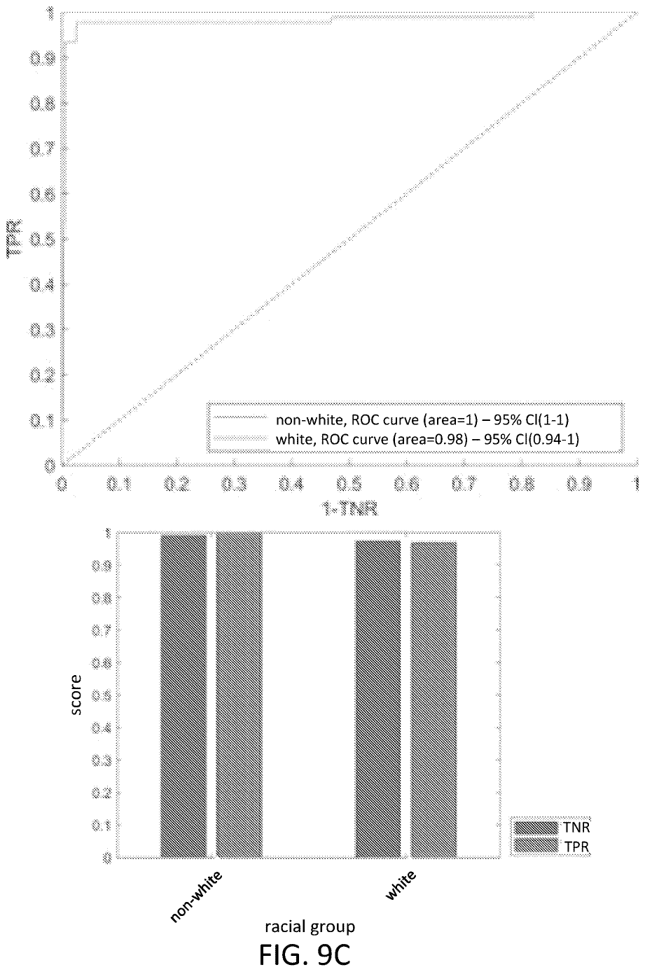

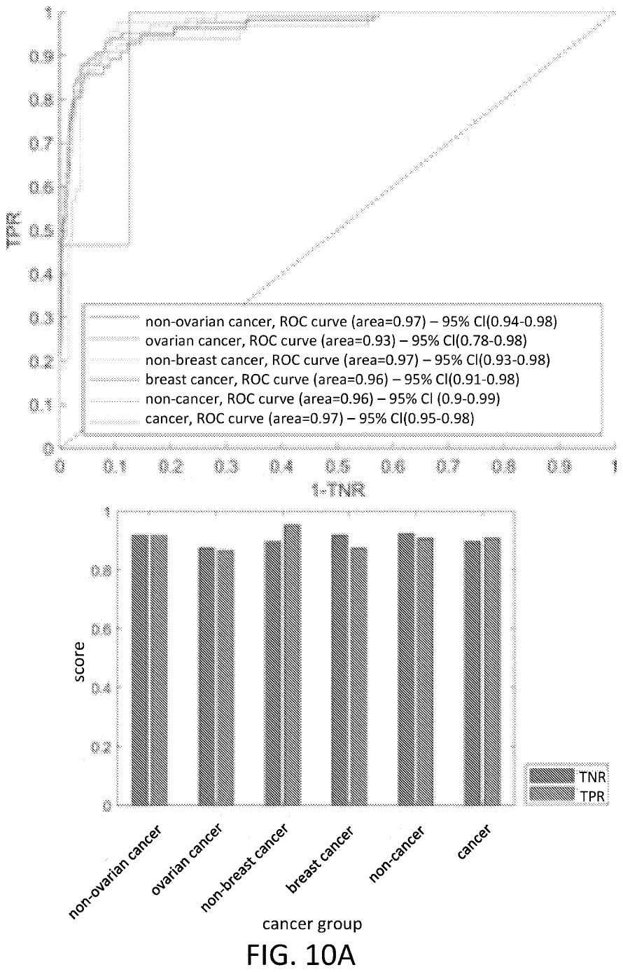

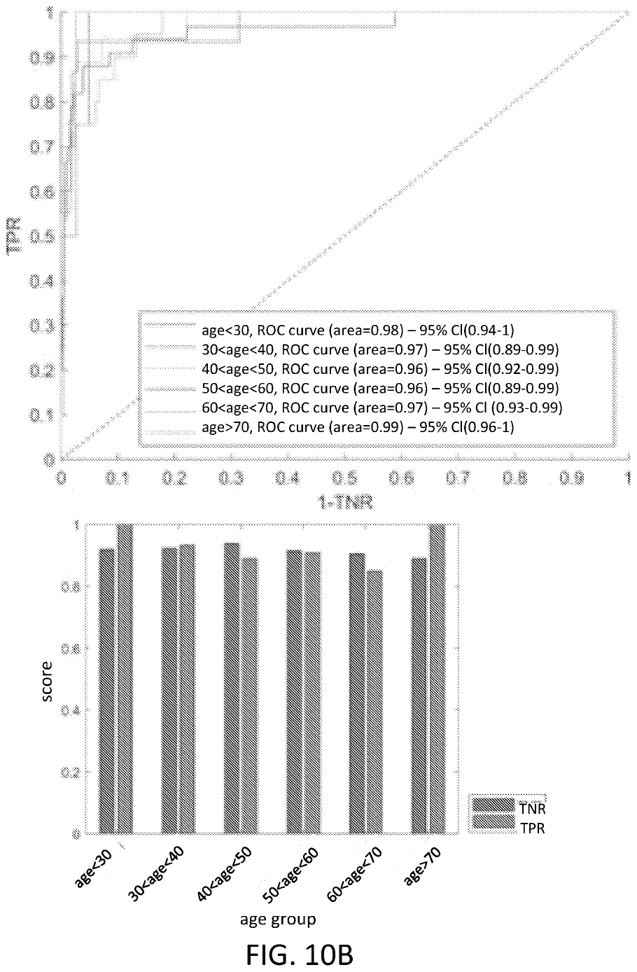

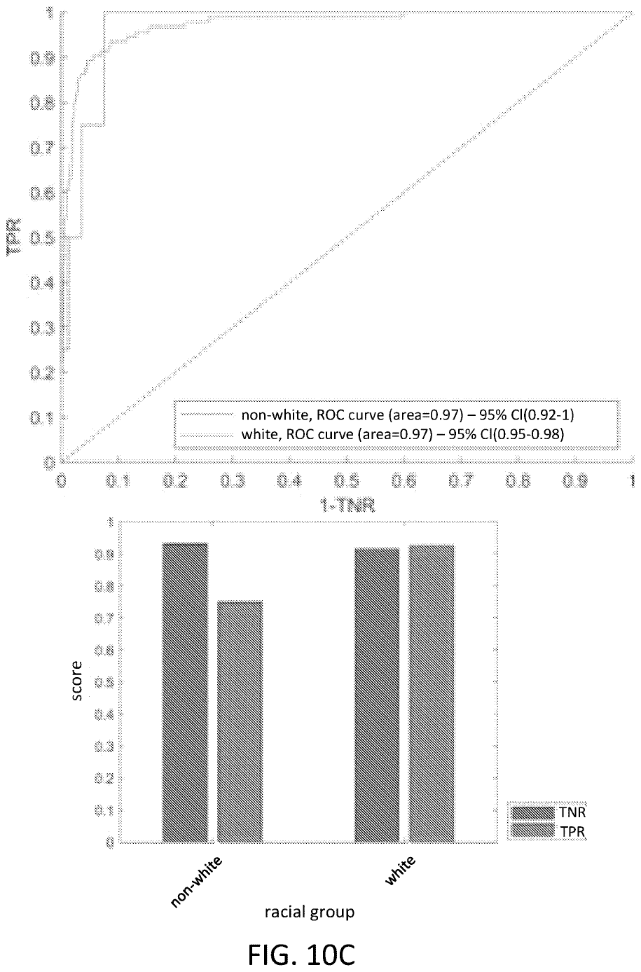

Subset analyses in which study subjects were grouped by race, age, or cancer status, were performed to determine the stability of the joint lasso model across different population subgroups. No differences were seen whether the subjects were split by ovarian or breast cancer, nor if using a collective “cancer” classifier that also included non-HBOC cancers such as thyroid, cervix, colon, and skin cancers (FIG. 9A). Likewise, if the study cohort is divided into 10-year blocks by age, the model performed similarly well among all age groups (FIG. 9B). Finally, the impact of race and ethnicity on model performance was examined. Due to small sample sizes for individual minority groups, race was split into “non-Hispanic white” or “all other.” The model performed similarly across racial groups (FIG. 9C). As with the larger model, classification within these subsets remained consistent when classified using the more limited set of model inputs described above (FIGS. 10A-10C).

Among the subjects considered in this study, 259 out of 1831 had genetic testing for BRCA mutations, while the untested subjects were presumed non-BRCA mutation carriers when training the joint lasso model. This may create bias or confounding effects in the classification results, especially by misclassifying true positives as presumed negatives. To address this, FIG. 7 shows a subgroup analysis of the performance of the joint lasso model of FIG. 6 and metadata component limited to subjects with known genetic testing results for BRCA mutations. The full data joint lasso model performance is largely retained (AUC=0.96, 95% CI 0.92-0.98) among patients with genetic testing. The baseline model (trained using metadata alone) only offers an AUC=0.57 (95% CI 0.48-0.63) among formally tested patients. Thus, when the models are evaluated on known tested subjects, after 10-fold cross-validation, the AUC score offered by the joint lasso model (96%) is 39% greater than the baseline model (57%) trained on metadata alone, which highlights the benefits of including miRNA in the joint model. A similar effect is seen using the limited data joint lasso model. See FIGS. 7C-7D.

BRCA1/2 Classifier Analysis Among Non-Carriers with Breast or Ovarian Cancer

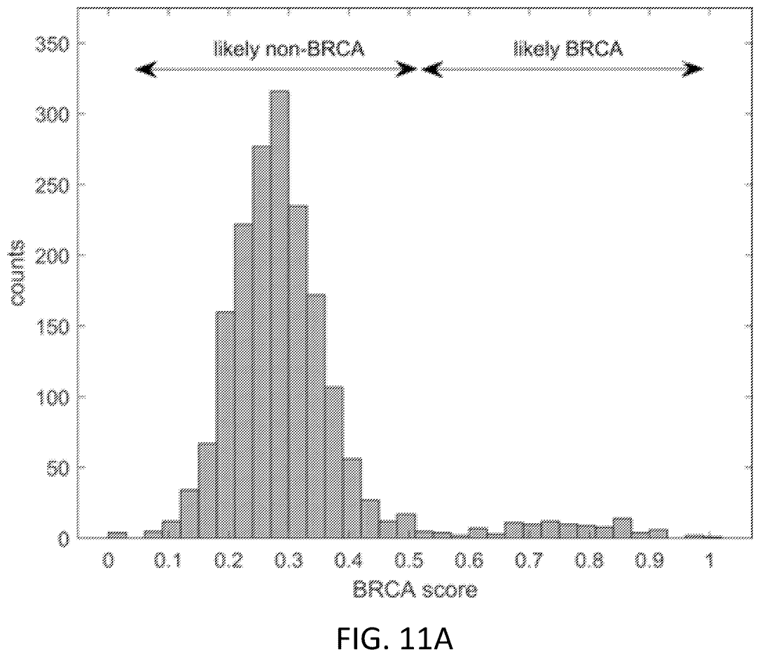

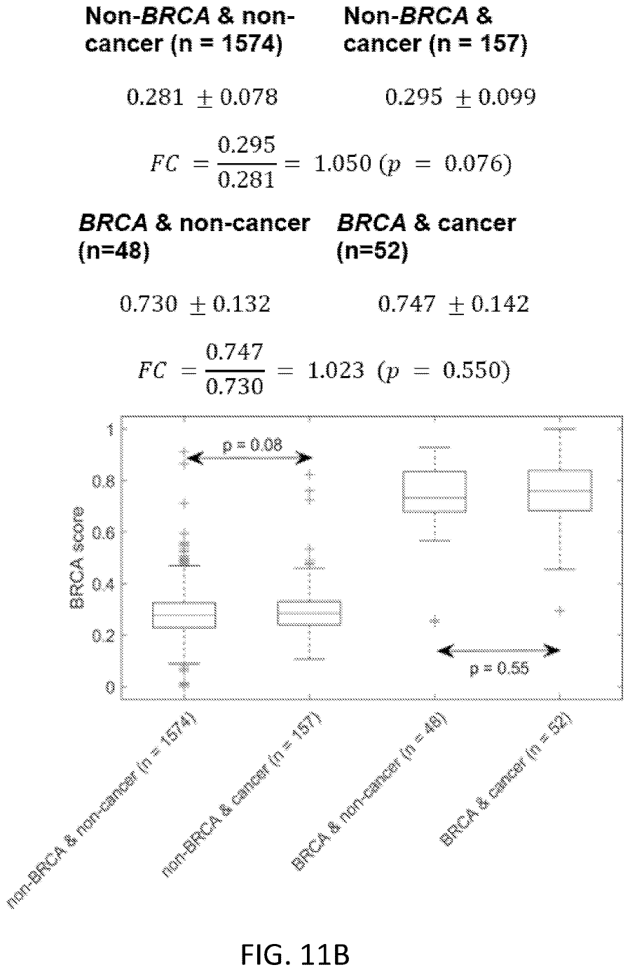

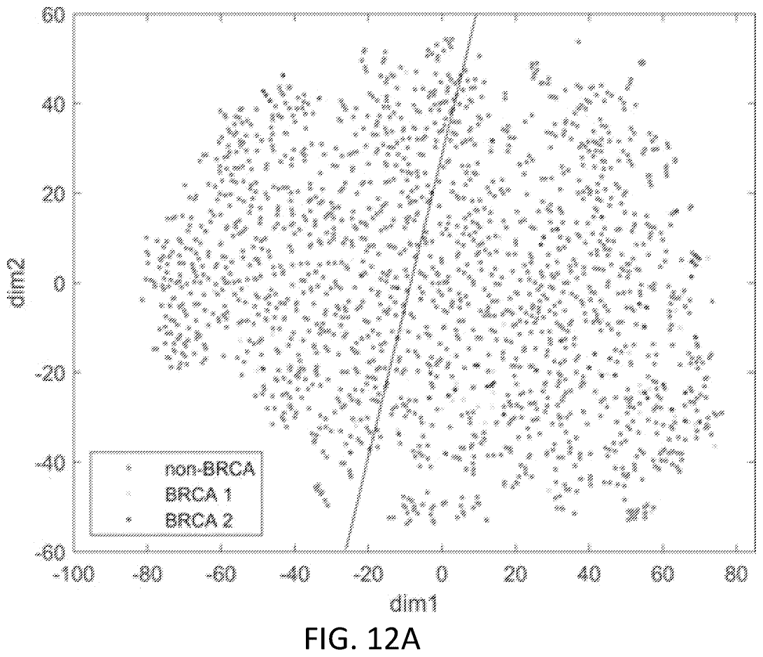

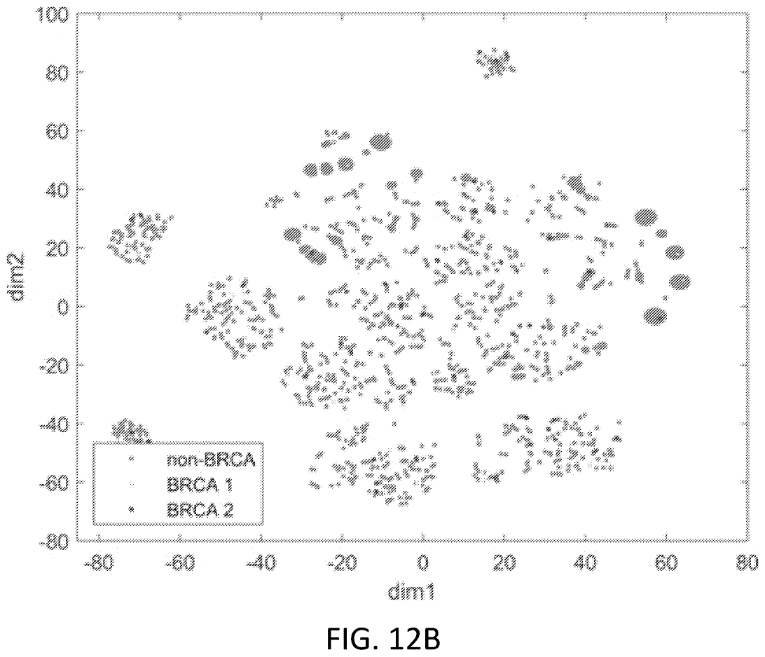

While germline BRCA1/2 mutations are identified in 13-15% of ovarian cancers and 3% of unselected breast cancers, an additional 5-7% of ovarian cancers and 3% of breast cancers harbor somatic BRCA1/2 mutations. Therefore, it is investigated whether the miRNA profiles of non-mutation carriers with breast or ovarian cancer might more closely resemble mutation carriers with or without cancer. Using the joint lasso model, mutation carriers and non-carriers had non-overlapping BRCA mutation probability scores (FIG. 11A). See the methods section for a detailed definition of the BRCA score. Cancer and non-cancer subjects had indistinguishable BRCA scores, when considered among mutation and non-mutation carriers separately (see the box plots of FIG. 11B). Overall, among all study subjects, non-cancer subjects had mean BRCA score 0.30, which is significantly smaller than that of cancer subjects (0.41, p<0.0001). The miRNA profiles could not distinguish BRCA1 from BRCA2 carriers (FIG. 12A).

### Clinical Application: Relation Between Model Predictions and Ovarian Cancer Risk

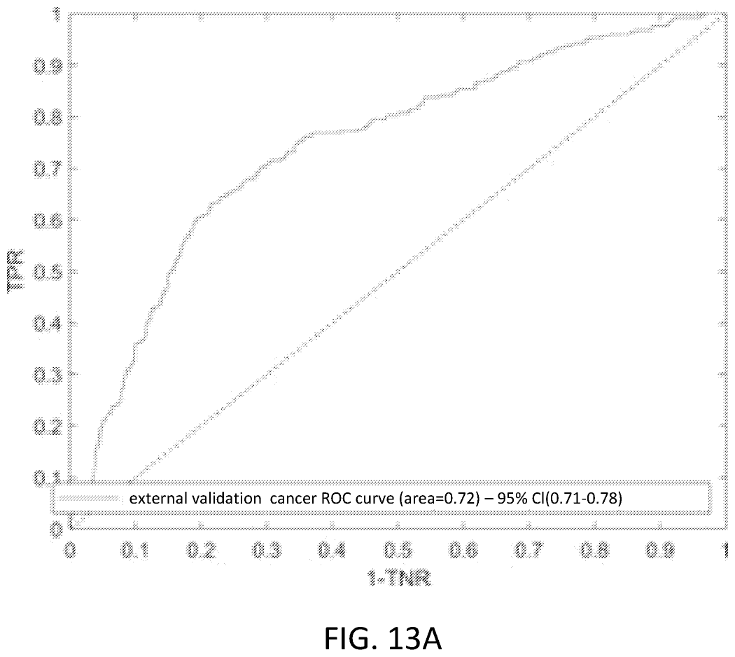

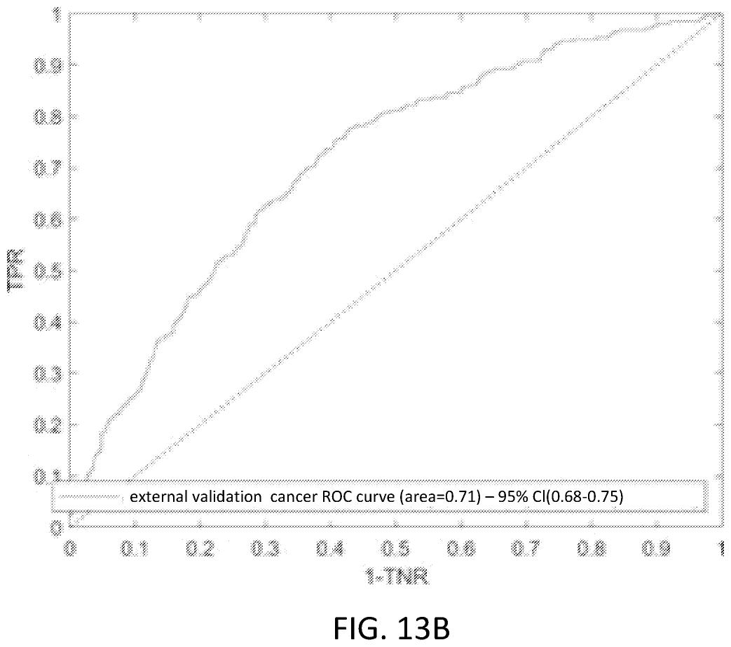

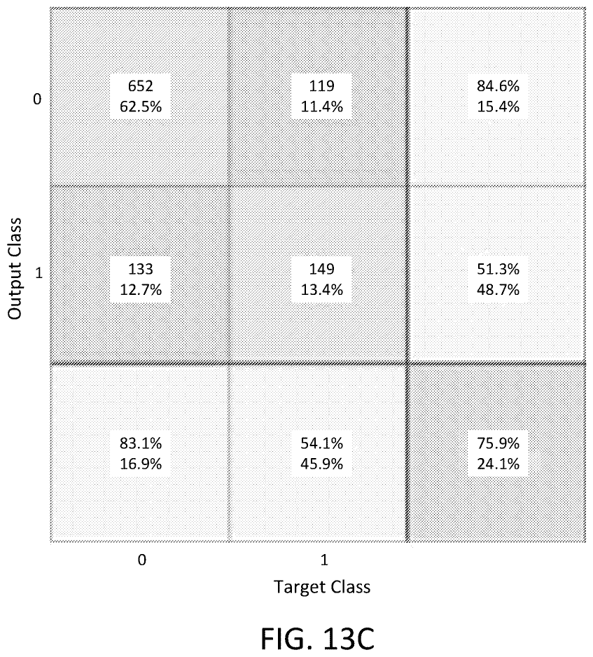

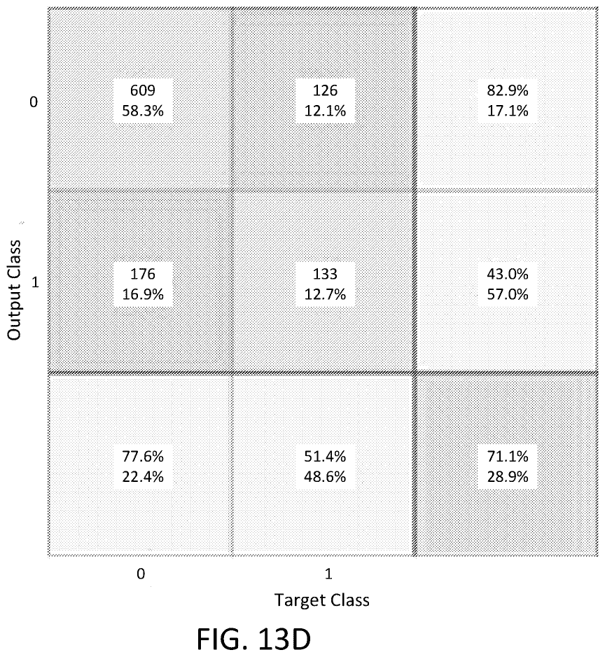

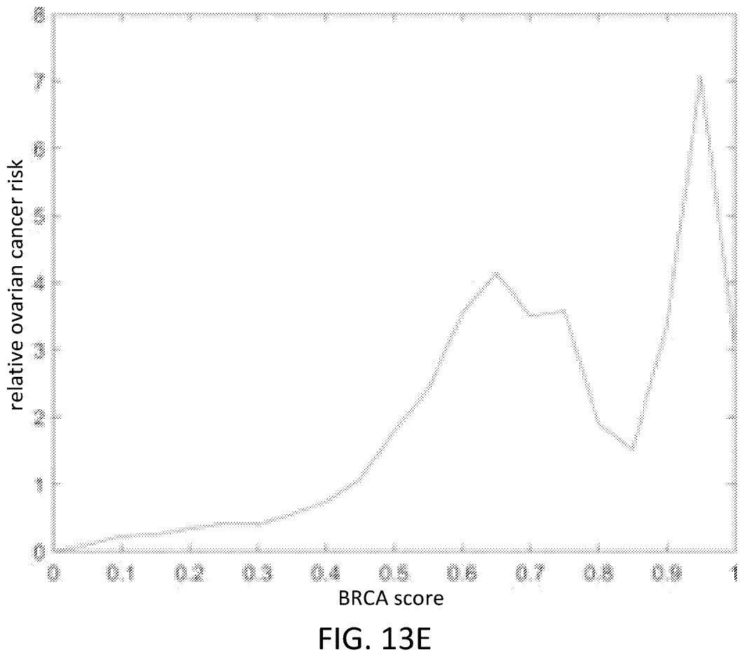

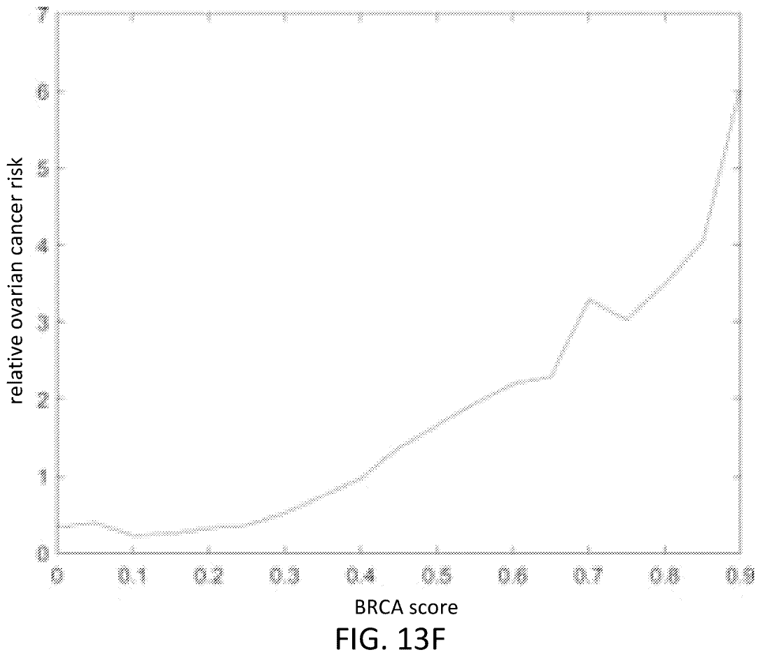

While having a screening test for BRCAness might improve the efficiency of genetic testing referrals, if the test is truly indicative of a predisposition to ovarian cancer, that should be reflected in the cancer risk observed in a population of unknown BRCA mutation status. To this end, the relation between BRCAness and ovarian cancer was investigated. In this instance, BRCAness is predicted by the joint lasso model; ovarian cancer is predicted on n=1044 samples collected as part of the PLCO cancer screening trial, consisting of 259 subjects later diagnosed with ovarian cancer and 785 matched controls. The cancer cases had their blood drawn at a range of times between 1 and 1814 days (up to 5 years) before cancer diagnosis. The BRCA mutation status of the PLCO samples is unknown, and thus the joint lasso model's ability to assess 5-year ovarian cancer risk directly is tested. See FIGS. 13A-13F. The external validation AUC scores are 0.74 (95% CI 0.71-0.78) and 0.71 (95% CI 0.67-0.75), using the full data and limited data joint lasso models, respectfully. Using the cancer thresholds calculated on the training data alone (e.g., t=0.04 for the full data model), the full and limited data models offer sensitivity and specificity scores of 54% and 83%, and 51% and 78%, respectively. See the confusion matrices in FIG. 13C and FIG. 13D for a full breakdown of the classification scores. To calculate the AUC in FIGS. 13A and 13B, compared the predicted BRCA score outputted by the joint lasso model is compared with true ovarian cancer diagnosis.

This explains the observed drop in AUC score (i.e., from 0.98 to 0.74), as predicted BRCA mutation status is being used as a surrogate marker for ovarian cancer risk. FIG. 13E and FIG. 13F show how the BRCA score relates to relative risk of ovarian cancer. A strong positive correlation between BRCA score and 5-year relative ovarian cancer risk using the full and limited data models is shown (R=0.80, p<0.0001, and R=0.92, p<0.0001, respectively). The cancer risk is calculated relative to the PLCO population. Thus, the joint lasso model (both the full and limited data variants) can be used successfully to identify long-term cancer risk. For example, if a subject presented with a BRCA score of 0.6, using the limited data model, their 5-year relative risk is approximately 2, which would indicate that closer monitoring and more regular cancer screening may be required, whereas a score of 0.9 would indicate a 5-year relative risk of approximately 6, which might prompt a discussion about risk-reducing surgical options.

### Discussion

This example presents a novel, first-pass screening test for BRCA1/2 mutations that combines a serum analyte (miRNA expression) with clinical metadata (personal and family history). A joint lasso model is used to reduce the dimensionality of the miRNA and metadata inputs to two dimensions. Following this, a shallow neural network was trained on the reduced dimension data to determine BRCA1/2 mutation probability. The proposed model offered an AUC score of 0.98 after 10-fold cross validation, which is a considerable improvement from the AUC of 0.89 previously reported using miRNAs alone, which were measured in the prior report using next generation sequencing. The model performance is evaluated across different racial, age, and cancer status groups and have shown the model largely retains its predictive capacity. The model performance is also tested with limited miRNA and metadata and found that a smaller set of inputs nearly replicated the full data model. This offers an advantage over previous work, in which model accuracy was not tested in terms of long-term cancer risk. Specifically, this work tests ability of the joint lasso model to assess long term (5-year) cancer risk on an external population collected as part of the PLCO cancer screening trial. The model offered an AUC score 0.73 when used to predict ovarian cancer directly, and the BRCA score outputted by the model had strong positive correlation with relative cancer risk (R=0.80, p<0.0001).

The current work is distinct from prior case-control studies that use miRNA expression to distinguish healthy subjects, or subjects with benign tumors, from those with cancer (see Chan, M., et al., 2013, Clin Cancer Res 19, 4477-4487; Yamamoto, Y. et al., 2020 Hepatol Commun 4, 284-297; Usuba, W. et al. 2019, Cancer Sci 110, 408-419; Elias, K. M., et al., 2017, Elife, 6). Such studies do not identify early indicators of cancer risk, as the models are trained on subjects who actively have cancer, most of which are late-stage cancers. As the outputs from the models are binary (e.g., cancer vs. control), they cannot account for competing cancer risks with shared risk factors, such as breast, ovarian, and uterine cancer. In contrast, BRCA mutation is a binary output, and thus the same competing risk issue is not encountered. Identifying these individuals at high risk for cancer who could benefit most from risk reduction surgeries or intensive surveillance strategies provides a more targeted approach to preventing cancer deaths.

These findings add to the literature demonstrating that BRCA mutations are associated with changes in miRNA expression. Tumor profiling has shown that both breast and ovarian cancer tissues from women with germline mutations in BRCA1/2 are distinct from sporadic tumors. In addition to identifying early indicators of cancer risk, this work is also able to identify circulating miRNA profiles in healthy women and women who have with cancer. This suggests a key role for miRNAs in the phenotype of HBOC, either as a compensatory response to defective DNA repair or as a downstream effect from the loss of homologous repair.

The current study has several strengths of note. First, a large clinical dataset is presented, reflecting the clinical and demographic diversity of women seen in actual clinical practice, not a focused subgroup of women participating in a cancer prevention trial. This stands in contrast to other miRNA based models from the literature focused on smaller sample sets and trained on miRNA expression from a single cancer type. Second, the described model works equally well among women with and without HBOC-associated cancers. This data includes healthy subjects, and subjects with a variety of cancers, such as breast, ovarian, skin, and cervical cancer. Finally, the use of miRNA expression technology and metadata to predict BRCA mutation offers significant efficiency and cost benefits when compared to conventional genetic testing for BRCA mutations using next generation sequencing. A first-pass screening based on miRNA and metadata has very high sensitivity, which can narrow down the wider population to a high-risk subgroup which can then be moved onto further screening (e.g., conventional genetic testing).

In conclusion, this work describes a highly robust and accurate model for BRCA mutation which can be performed at greatly reduced cost and increased efficiency compared to conventional genetic testing. The result may improve the ability to identify individuals at risk for HBOC and to implement new cancer prevention and risk management strategies.

### Methods

### Study Population

Serum samples were collected from study subjects participating in the Mass General Brigham Biobank enrolled between 2012 and 2022. Subjects were selected based on a documented visit with a gynecologist in the electronic health record. Samples were collected under Mass General Brigham IRB protocol 2018P001680. Demographic characteristics and medical histories were abstracted from the medical record using manual chart review. Race and ethnicity were self-identified within the medical record, and for purposes of analysis were defined as white, non-Hispanic vs non-white. Mutation carriers were identified by a documented germline genetic testing report in the electronic health record.

miRNA Profiling

miRNA profiles were generated for 179 different miRNA species using Fireplex® probes (Abcam, Cambridge, MA) and measured in mean fluorescence units (MFI) using Guava Easycyte 5HT flow cytometers (Luminex, Austin, TX) according to the manufacturer's instructions. The panel of miRNAs was optimized to capture serum miRNAs detectable in at least 50% of samples based on a prior next generation sequencing study. Twenty-five μL of serum were utilized for each sample. As the assay can profile up to 68 miRNAs per well on a 96-well plate, each biologic sample was distributed across three assay panels to construct the full 179 miRNA profile, with some overlap between panels to allow for quality control. Each plate also included a well of pooled human serum, water controls, and spike-in reference miRNAs. The panel includes off-species control probes targeting C. elegans miRNAs to establish background signal levels. Samples were processed using a STARlet liquid handling robot (Hamilton Robotics, Franklin, MA) and analyzed using the FirePlex® Analysis Workbench software (abcam.com/FireflyAnalysisSoftware). Technical replicates were not performed, but the coefficient of variation between individual miRNA values for the same sample averages less than 20%. Any outlier samples in terms of quality control using the off-species miRNAs and reference miRNAs were repeated.

### Joint Lasso Model

A joint lasso Dimensionality Reduction (DR) approach combined with a linear classification model is used to classify BRCA vs non-BRCA. Let X₁∈ [symbol] ^{n×p}^{₁} be a matrix of normalized miRNA expression values, where n is the number of samples and p₁ the number of miRNAs, and let X₂∈ [symbol] ^{n×p}^{₂} be a matrix of metadata, where p₂ is the number of metadata variables. For example, the column of X₂ which corresponds to BRCA family history (see Table 8) is a binary vector (i.e., its entries are 0 or 1), where 0 indicates no BRCA family history, and 1 indicates BRCA family history. Let Y∈{0,1}ⁿ be a binary vector of class labels, where 0 indicates non-BRCA, and 1 indicates BRCA. A joint lasso is used to reduce the dimension of the miRNA and metadata. Specifically, the aim is to find:

$$( A , 1 ) \frac{argmin}{{v}_{1} ∈ {ℝ}^{{p}_{1}}} {‖ {X}_{1} {v}_{1} - Y ‖}_{2}^{2} + {β}_{1} {‖ {v}_{1} ‖}_{1} ,$$

$$and$$

$$\frac{argmin}{{v}_{1} ∈ {ℝ}^{{p}_{2}}} {‖ {X}_{2} {v}_{2} - Y ‖}_{2}^{2} + {β}_{2} {‖ {v}_{2} ‖}_{1} ,$$

where ∥v∥₁=Σᵢ|vᵢ| denotes L¹ norm, and β₁; β₂>0 are regularization parameters which control the level of sparsity in v₁; v₂, respectively. The lasso models were fit using the “lasso” Matlab function. Once v₁ and v₂ are determined, the miRNA and metadata are mapped to two-dimensional space:

$$[ {X}_{1} , {X}_{2} ] \to X = [ {X}_{1} {v}_{1} , {X}_{2} {v}_{2} ] .$$

Then, to classify subjects as BRCA or non-BRCA, a linear classification model is trained on X using the equation:

$$P ( y = j \vert x ) = \frac{{e}^{{x}^{T} {w}_{j} + {b}_{j}}}{{\sum }_{i = 0}^{{n}_{c} - 1} {e}^{{x}^{T} {w}_{i} + {b}_{i}}}$$

where j∈{0,1} is the class label, x∈ [symbol] ² is a sample in reduced dimension space (i.e., one row of X), and the (wᵢ, bᵢ) are weights and biases to be trained. Here y denotes the class label assigned to x. A subject is then classified as having BRCA mutation if P(j=1,x)>t, where t∈[0,1] is the BRCA threshold. The classifier was trained using the “trainSoftmaxLayer” Matlab function. To validate the joint lasso model, 10-fold cross validation is used. The hyperparameters, β₁ and β₂, are chosen using nested 10-fold validation on each training fold.

A “BRCA score” is discussed above. The BRCA score is defined bₛ=x^{T}w₁+b₁, which is then translated and scaled to be within the range [0,1], for better interpretability.

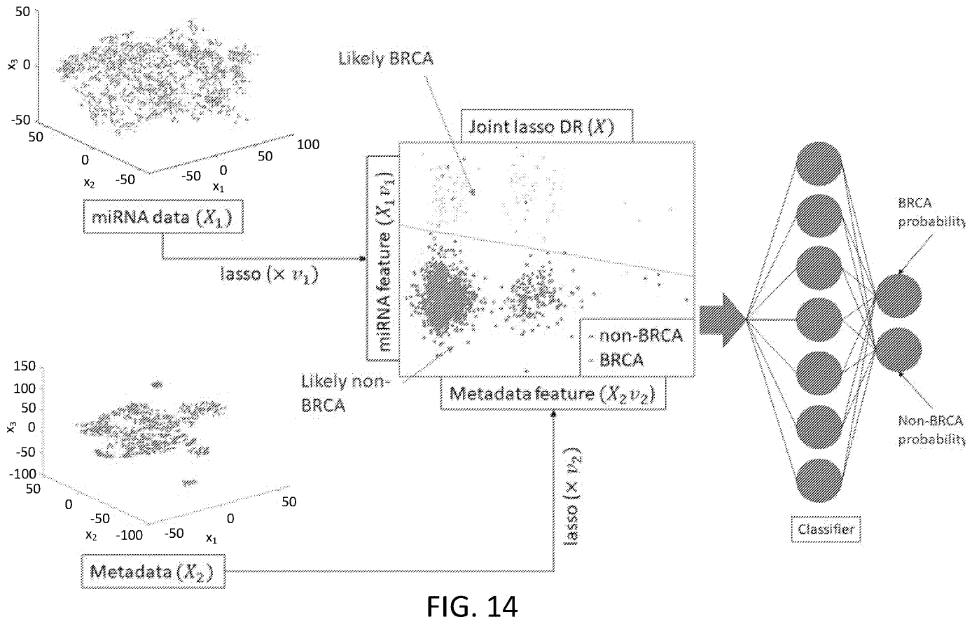

FIG. 14 shows a schematic of a classification procedure. The left-hand side shows 3-D t-distributed Stochastic Neighbor Embedding (TSNE) plots of the miRNA and metadata, in order to visualize the two data sets and show how the BRCA and non-BRCA subjects separate. The plots indicate a mild, linear separation of the BRCA and non-BRCA classes. The center shows the result of the joint lasso dimension reduction (i.e., X). The miRNA feature (X₁v₁) is shown on the y axis, and the metadata feature (X₂v₂) is on the x axis. There is significant linear separation between BRCA and non-BRCA subjects in the reduced dimension space. The X space is split into two parts: one the likely BRCA group, and the other the likely non-BRCA group. Once the miRNA and metadata are projected into 2-D space as in the central scatter plot of FIG. 14, the classifier (illustrated on the right-hand of FIG. 14) assigns a probability to BRCA (i.e., P(j=1,x)) and non-BRCA (P(j=0,x)) as described above. Then, a threshold t∈[0,1] is defined, and the subject is classified as a BRCA mutation carrier if their mutation probability is greater than t, and conversely as a non-carrier if the probability of BRCA is less than or equal to t. The parameter t allows us to decide whether to favor model specificity or sensitivity. For example, setting t closer to zero gives more weight to sensitivity, and vice-versa.

What is claimed is:

1. A method of predicting a lifetime risk of developing one or more cancers, in a subject suspected of having a BRCA1 or BRCA2 mutation, comprising:
(a) obtaining a sample collected from the subject;
(b) determining an amount of a circulating microRNA selected from the group consisting of: hsa-miR-20b-5p (SEQ ID NO: 4), hsa-miR-19b-3p (SEQ ID NO: 3), hsa-let-76-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19);
(c) comparing the amounts of circulating microRNA as determined in step (b) to a statistical model; and
(d) predicting the lifetime risk of the subject developing one or more cancers based on identifying the presence of at least one mutation in the BRCA1 or BRCA2 gene based upon the amounts of one or more of the circulating miRNAs.

2. The method according to claim 1, wherein step (b) comprises determining the amount of ten microRNAs including hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), and hsa-miR-375-3p (SEQ ID NO: 10) in the sample.

3. The method according to any one of claim 1 or 2, wherein the cancer comprises one or more of breast cancer, ovarian cancer, pancreatic, or prostate cancer.

4. The method according to any one of claim 1 or 2, wherein the sample is selected from a sample of blood.

5. The method according to any one of claim 1 or 2, wherein the blood sample is selected from the group consisting of plasma, serum, and whole blood.

6. The method according to any one of claim 1 or 2, wherein the statistical model comprises one or more models selected from the group consisting of linear discriminant analysis, logistic regression mode, multivariate adaptive regression splines, naïve Bayes, neural network, support vector machine, functional tree, LAD tree, Bayesian network, elastic net regression, and random forest.

7. The method according to claim 6, wherein the statistical model comprises a logistic regression model.

8. The method according to claim 6, wherein the statistical model comprises dimensionality reduction techniques.

9. The method according to claim 6, wherein the statistical model comprises joint lasso dimensionality reduction techniques.

10. The method according to claim 6, wherein the model comprises joint lasso dimensionality reduction techniques and sparse machine learning techniques.

11. The method according to any one of claim 1 or 2, wherein step (b) and/or step (d) are performed using RNA sequencing.

12. The method according to any one of claim 1 or 2, further comprising performing genetic testing or genetic counseling.

13. The method according to any one of claim 1 or 2, further comprising administering a treatment to the subject, wherein the treatment is selected from the group consisting of surgery, chemotherapy, immunotherapy, radiation therapy, hormone therapy, and stem cell transplant.

14. The method according to any one of claim 1 or 2, wherein the subject is female.

15. A method of identifying a subject as having a BRCA mutation, the method comprising:
(a) obtaining a sample collected from the subject;
(b) determining an amount of a circulating microRNA selected from the group consisting of: hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19).
(c) comparing the amounts of circulating microRNA as determined in step (b) to a statistical model;
(d) predicting a lifetime risk of the subject developing one or more cancers based on identifying the presence of at least one mutation in the BRCA1 or BRCA2 gene; and
(e) performing genetic testing on the subject.

16. The method according to claim 15, wherein step (b) comprises determining the amount of ten microRNAs of hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), and hsa-miR-375-3p (SEQ ID NO: 10) in the sample.

17. The method according to any one of claim 15 or 16, further comprising monitoring the subject for a BRCA associated cancer.

18. The method according to any one of claim 15 or 16, wherein the BRCA associated cancer comprises one or more of breast cancer, ovarian cancer, pancreatic, or prostate cancer.

19. The method according to any one of claims 15-18, further comprising, administering a treatment to the subject, wherein the treatment is selected from the group consisting of surgery, chemotherapy, immunotherapy, radiation therapy, hormone therapy, and stem cell transplant.

20. The method according to any one of claim 15 or 16, wherein the statistical model comprises one or more models selected from the group consisting of linear discriminant analysis, logistic regression mode, multivariate adaptive regression splines, naïve Bayes, neural network, support vector machine, functional tree, LAD tree, Bayesian network, elastic net regression, and random forest.

21. The method according to claim 20, wherein the statistical model comprises a logistic regression model.

22. The method according to claim 20, wherein the statistical model comprises dimensionality reduction techniques.

23. The method according to claim 20, wherein the model comprises joint lasso dimensionality reduction techniques.

24. The method according to claim 20, wherein the model comprises joint lasso dimensionality reduction techniques and sparse machine learning techniques.

25. A method of treating a patient suspected of having a BRCA associated cancer, the method comprising:
(a) obtaining a sample collected from the subject;
(b) determining the amounts of a circulating microRNA selected from the group consisting of: hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19);
(c) comparing the amounts of circulating microRNA as determined in step (b) to a statistical model;
(d) predicting a lifetime risk of the subject developing one or more cancer based on identifying the presence of at least one mutation in the BRCA1 or BRCA2 gene; and
(e) administering to the subject a treatment selected from the group consisting of surgery, chemotherapy, immunotherapy, radiation therapy, hormone therapy, and stem cell transplant.

26. A kit comprising at least one test probe capable of specifically hybridizing to a microRNA selected from the group consisting of hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), hsa-miR-375-3p (SEQ ID NO: 10); hsa-miRNA-106b-5p (SEQ ID NO: 11); hsa-miRNA-134-5p (SEQ ID NO: 12), hsa-miRNA-493-5p (SEQ ID NO: 13), hsa-miRNA-500a-3p (SEQ ID NO: 14), hsa-miR-1273 h-3p (SEQ ID NO: 15), hsa-miR-4433a-3p (SEQ ID NO: 16), hsa-miR-4433b-5p (SEQ ID NO: 17), hsa-miR-485-3p (SEQ ID NO: 18), and has-miR-1304-3p (SEQ ID NO: 19).

27. The kit of claim 26, wherein at least one of the probes comprises a detectable label.

28. The kit according to any one of claim 26 or 27, further comprising a reagent for reverse transcription of a microRNA molecule.

29. The kit according to any one of claims 26-28, wherein the at least one test probe is capable of specifically hybridizing to microRNAs including hsa-miR-20b-5p (SEQ ID NO: 4), hsa-miR-19b-3p (SEQ ID NO: 3), hsa-let-7b-5p (SEQ ID NO: 1), hsa-miR-320b (SEQ ID NO: 8), hsa-miR-139-3p (SEQ ID NO: 6), hsa-miR-30d-5p, (SEQ ID NO 5), hsa-miR-17-5p (SEQ ID NO: 2), hsa-miR-182-5p (SEQ ID NO: 7), hsa-miR-421 (SEQ ID NO: 9), and hsa-miR-375-3p (SEQ ID NO: 10).
